(19) United States
(12) Patent Application Publication
(10) Pub. No.: US 2009/0131494 A1
(43) Pub. Date: May 21, 2009

(54) Non-Nucleoside Reverse Transcriptase Inhibitors

(76) Inventors: Theresa M. Williams, Harleysville, PA (US); Xu-Fang Zhang, Dresher, PA (US)

Correspondence Address: MERCK AND CO., INC, P O BOX 2000, RAHWAY, NJ, 07065-0907, US

(21) Appl. No.: 11/990,060

(22) PCT Filed: Aug. 7, 2006
(86) PCT No.: PCT/US06/30680

§ 371 Date: Feb. 5, 2008

Related U.S. Application Data
(60) Provisional application No. 60/707,337, filed on Aug. 11, 2005.

(51) Int. Cl. A61K 31/4178; A61K 31/404; C07D 403/12; A61P 31/18; C07D 209/42

(52) U.S. Cl. 514/397; 548/484; 514/418; 548/312.1

(57) ABSTRACT

Certain 1H-indole-2-carboxylates and -2-carboxamides are HIV reverse transcriptase inhibitors. These indole compounds and their pharmaceutically acceptable salts are useful in the inhibition of HIV reverse transcriptase, the prophylaxis and treatment of infection by HIV and in the prophylaxis, delay in the onset, and treatment of AIDS. The compounds and their salts can be employed as ingredients in pharmaceutical compositions, optionally in combination with other antivirals, immunomodulators, antibiotics or vaccines.

### FIELD OF THE INVENTION

The present invention is directed to certain indoles and their pharmaceutically acceptable salts and their use for the inhibition of HIV reverse transcriptase, the prophylaxis and treatment of HIV infection and HIV replication, and the prophylaxis, delay in the onset of and treatment of AIDS.

### BACKGROUND OF THE INVENTION

The retrovirus designated human immunodeficiency virus (HIV), particularly the strains known as HIV type-1 (HIV-1) and type-2 (HIV-2) viruses, have been etiologically linked to the immunosuppressive disease known as acquired immunodeficiency syndrome (AIDS). HIV seropositive individuals are initially asymptomatic but typically develop AIDS related complex (ARC) followed by AIDS. Affected individuals exhibit severe immunosuppression which makes them highly susceptible to debilitating and ultimately fatal opportunistic infections. Replication of HIV by a host cell requires integration of the viral genome into the host cell's DNA. Since HIV is a retrovirus, the HIV replication cycle requires transcription of the viral RNA genome into DNA via an enzyme know as reverse transcriptase (RT).

Reverse transcriptase has three known enzymatic functions: The enzyme acts as an RNA-dependent DNA polymerase, as a ribonuclease, and as a DNA-dependent DNA polymerase. In its role as an RNA-dependent DNA polymerase, RT transcribes a single-stranded DNA copy of the viral RNA. As a ribonuclease, RT destroys the original viral RNA and frees the DNA just produced from the original RNA. And as a DNA-dependent DNA polymerase, RT makes a second, complementary DNA strand using the first DNA strand as a template. The two strands form double-stranded DNA, which is integrated into the host cell's genome by the integrase enzyme.

It is known that compounds that inhibit enzymatic functions of HIV RT will inhibit HIV replication in infected cells. These compounds are useful in the prophylaxis or treatment of HIV infection in humans. Among the compounds approved for use in treating HIV infection and AIDS are the RT inhibitors 3′-azido-3′-deoxythymidine (AZT), 2′,3′-dideoxyinosine (ddI), 2′,3′-dideoxycytidine (ddC), d4T, 3TC, nevirapine, delavirdine, efavirenz and abacavir.

While each of the foregoing drugs is effective in treating HIV infection and AIDS, there remains a need to develop additional HIV antiviral drugs including additional RT inhibitors. A particular problem is the development of mutant HIV strains that are resistant to the known inhibitors. The use of RT inhibitors to treat AIDS often leads to viruses that are less sensitive to the inhibitors. This resistance is typically the result of mutations that occur in the reverse transcriptase segment of the pol gene. The continued use of antiviral compounds to prevent HIV infection will inevitably result in the emergence of new resistant strains of HIV. Accordingly, there is a particular need for new RT inhibitors that are effective against mutant HIV strains.

The following references are of interest as background:
Williams et al., J. Med. Chem. 1993, vol. 36, pp. 1291-1294 discloses 5-chloro-3-(phenylsulfonyl)indole-2-carboxamide as a non-nucleoside inhibitor of HIV-1 reverse transcriptase. Related N-methylcarboxamide and methyl carboxylate compounds are also disclosed.
Young et al., Bioorg. & Med. Chem. Letters 1995, vol. 5, pp. 491-496 discloses certain 2-heterocyclic indole-3-sulfones as inhibitors of HIV-1 reverse transcriptase.
GB 2,282,808 discloses certain 2-heterocyclic indole-3-sulfones as inhibitors of HIV reverse transcriptase and its resistant varieties.
U.S. Pat. No. 5,527,819 discloses certain 2-acyl substituted indole-3-sulfones as inhibitors of HIV reverse transcriptase.
WO 02/083216 A1 and WO 2004/014364 A1 each disclose certain substituted phenylindoles for the treatment of HIV.
Silvestri et al., J. Med. Chem. 2003, vol. 46, pp. 2482-2493 discloses indolyl aryl sulfones which are active against HIV-1 having nnRTI resistance mutations. Corresponding indole-2-carboxylates are disclosed in the preparative schemes.
Silvestri et al., J. Med. Chem. 2004, vol. 47, pp. 3892-3896 discloses simple, short peptide derivatives of 5-chloro-3-(3,5-dimethylphenylsulfonyl)indole-2-carboxamide. The derivatives showed activity against wild type HIV-1 and nnRTI-resistant mutants in cell-based assays. The reference also discloses ethyl 5-chloro-3-(3,5-dimethylphenylsulfonyl)indole-2-carboxylate and ethyl 5-chloro-3-(3,5-dimethylphenylthio)indole-2-carboxylate as intermediates in the preparation of the corresponding carboxamide. The reference also discloses 5-chloro-3-phenylsulfonylindole-2-carboxamide.

### SUMMARY OF THE INVENTION

The present invention is directed to certain 1H-indole-2-carboxylates and -2-carboxamides and their use in the inhibition of HIV reverse transcriptase, the prophylaxis of infection by HIV, the treatment of infection by HIV, and the prophylaxis, treatment, and delay in the onset of AIDS and/or ARC. The present invention includes compounds of Formula I and pharmaceutically acceptable salts thereof:

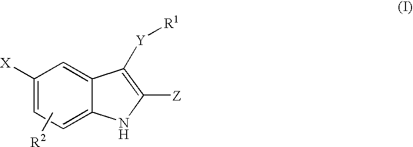

wherein:

### X is:

(1) halogen,
(2) CN,
(3) NO₂,
(4) C(O)R^{A},
(5) C(O)OR^{A},
(6) C(O)N(R^{A})R^{B},
(7) SR^{A},
(8) S(O)R^{A},
(9) S(O)₂R^{A},
(10) S(O)₂N(R^{A})R^{B},
(11) N(R^{A})R^{B},
(12) N(R^{A})S(O)₂R^{B},
(13) N(R^{A})C(O)R^{B},
(14) N(R^{A})C(O)ORB,
(15) N(R^{A})S(O)₂N(R^{A})R^{B},
(16) OC(O)R^{A},
(17) OC(O)N(R^{A})R^{B},
(18) N(R^{A})C(O)N(R^{A})R^{B},
(19) C₁₋₆ alkyl,
(20) C₁₋₆ haloalkyl,
(21) C₂₋₆ alkenyl,
(22) C₂₋₆ alkynyl,
(23) OH,
(24) O—C₁₋₆ alkyl,
(26) O—C₁₋₆ haloalkyl, or
(27) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)R^{A}, OC(O)N(R^{A})R^{B}, or N(R^{A})C(O)N(R^{A})R^{B},
(28) C₃₋₈ cycloalkyl, or
(29) C₁₋₆ alkyl substituted with C₃₋₈ cycloalkyl;

### Y is S, S(O), or S(O)₂;

### Z is C(O)N(H)R^{C} or C(O)OR^{D};

### R^{C} is:

(1) H,
(2) C₁₋₆ alkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)R^{A}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(4) O—C₁₋₆ alkyl,
(5) CycA,
(6) AryA,
(7) HetA,
(8) HetR, or
(9) C₁₋₆ alkyl substituted with CycA, AryA, HetA, or HetR;

### R^{D} is:

(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)R^{A}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule, or
(3) C₁₋₆ alkyl substituted with CycA, AryA, HetA, or HetR;
   CycA is C₃₋₈ cycloalkyl which is optionally substituted with a total of from 1 to 6 substituents, wherein:

(i) from zero to 6 substituents are each independently:
(1) halogen,
(2) CN
(3) C₁₋₆ alkyl,
(4) OH,
(5) O—C₁₋₆ alkyl,
(6) C₁₋₆ haloalkyl, or
(7) O—C₁₋₆ haloalkyl, and

(ii) from zero to 2 substituents are each independently:
(1) CycE,
(2) AlyE,
(3) O-AryE,
(4) HetE,
(5) HetF, or
(6) C₁₋₆ alkyl substituted with CycE, AryE, O-AryE, HetE, or HetF;
   AryA is aryl which is optionally substituted with a total of from 1 to 6 substituents, wherein:

(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₁₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)R^{A},
(16) OC(O)N(R^{A})R^{B},
(17) SR^{A},
(18) S(O)R^{A},
(19) S(O)₂R^{A},
(20) S(O)₂N(R^{A})R^{B},
(21) N(R^{A})S(O)₂R^{B},
(22) N(R^{A})S(O)₂N(R^{A})R^{B},
(23) N(R^{A})C(O)R^{B},
(24) N(R^{A})C(O)N(R^{A})R^{B},
(25) N(R^{A})C(O)—C(O)N(R^{A})R^{B}, or
(26) N(R^{A})CO₂R^{B}, and

(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
   HetA is heteroaryl which is optionally substituted with a total of from 1 to 6 substituents, wherein:

(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₁₆ alkyl,
(4) C₁₋₁₆ haloalkyl,
(5) O—C₁₋₆ haloalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)R^{A},
(17) OC(O)N(R^{A})R^{B},
(18) SR^{A},
(19) S(O)R^{A},
(20) S(O)₂R^{A},
(21) S(O)₂N(R^{A})R^{B},
(22) N(R^{A})S(O)₂R^{B},
(23) N(R^{A})S(O)₂N(R^{A})R^{B},
(24) N(R^{A})C(O)R^{B},
(25) N(R^{A})C(O)N(R^{A})R^{B},
(26) N(R^{A})C(O)—C(O)N(R^{A})R^{B}, or
(27) N(R^{A})CO₂R^{B}, and

(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
   HetR is a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, and wherein the saturated or mono-unsaturated heterocyclic ring is optionally substituted with a total of from 1 to 4 substituents, wherein:
(i) from zero to 4 substituents are each independently halogen, CN, C₁₋₆ alkyl, OH, oxo, C(O)R^{A}, C(O)₂R^{A}, S(O)R^{A}, SR^{A}, S(O)₂R^{A}, O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl; and
(ii) from zero to 2 substituents are each independently CycE, HetE, AryE, HetF, or C₁₋₆ alkyl substituted with CycE, AryE, HetE, or HetF;

### R¹ is:

(1) C₁₋₈ alkyl,
(2) C₁₋₈ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(3) C₁₋₈ haloalkyl,
(4) C₂₋₈ alkenyl,
(5) CycB,
(6) HetS, or
(7) C₁₋₈ alkyl substituted with CycB or HetT;
   CycB is C₃₋₈ cycloalkyl or C₅₋₈ cycloalkenyl, wherein the cycloalkyl or cycloalkenyl is optionally substituted with a total of from 1 to 6 substituents, wherein:

(i) from zero to 6 substituents are each independently:
(1) halogen,
(2) CN
(3) C₁₋₆ alkyl,
(4) OH,
(5) O—C₁₋₁₆ alkyl,
(6) C₁₋₆ haloalkyl,
(7) O—C₁₋₆ haloalkyl, or
(8) C(O)OR^{A}, and

(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
   HetS is a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, wherein the saturated or mono-unsaturated heterocyclic ring is attached to the rest of the molecule via a ring carbon, and wherein the saturated or mono-unsaturated heterocyclic ring is optionally substituted with a total of from 1 to 4 substituents, wherein:
(i) from zero to 4 substituents are each independently halogen, CN, C₁₋₆ alkyl, OH, oxo, S(O)R^{A}, SR^{A}, S(O)₂R^{A}, O—C₁₋₆ alkyl, C(O)O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl; and
(ii) from zero to 2 substituents are each independently CycE, HetE, AryE, HetF, or C₁₋₆ alkyl substituted with CycE, AryE, HetE, or HetF;
   HetT independently has the same definition as HetR;
   R² is H or independently has the same definition as X;
   each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
   each heteroaryl is independently (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂;
   each CycE is independently C₃₋₈ cycloalkyl which is optionally substituted with from 1 to 4 substituents each of which is independently halogen, C₁₋₆ alkyl, OH, O—C₁₋₆ alkyl, C₁₋₆ haloalkyl, or O—C₁₋₆ haloalkyl;
   each AryE is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B};
   each HetE is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently halogen, C₁₋₆ alkyl, C₁₋₆ haloalkyl, O—C₁₋₆ alkyl, O—C₁₋₆ haloalkyl, OH, N(R^{A})R^{B}, N(R^{A})C(O)N(R^{A})R^{B}, or N(R^{A})CO₂R^{B};
   each HetF is independently a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, and wherein the saturated or mono-unsaturated heterocyclic ring is optionally substituted with a total of from 1 to 4 substituents, each of which is independently halogen, CN, C₁₋₆ alkyl, OH, oxo, O—C₁₋₆ alkyl, C₁₋₆ haloalkyl, or O—C₁₋₆ haloalkyl;
   each R^{A} is independently H or C₁₋₆ alkyl; and

each R^{B} is independently H or C₁₋₆ alkyl; and with the proviso that:

(A) when:
(i) X is Cl, F, Br, NO₂, CN, OH, O—C₁₋₃ alkyl, NH₂, N(H)—C₁₋₃ alkyl, N(C₁₋₃ alkyl)₂, NHSO₂—C₁₋₃ alkyl, or NHC(O)—C₁₋₃ alkyl,
(ii) R² is H, and
(iii) Z is:
   (a) C(O)N(H)R^{C}, wherein R^{C} is:
      (1) H,
      (2) C₁₋₆ alkyl,
      (3) O—C₁₋₅ alkyl,
      (4) C₁₋₅ alkyl substituted with OH, O—C₁₋₅ alkyl, OC(O)H, OC(O)—C₁₋₃ alkyl, CO₂H, C(O)O—C₁₋₃ alkyl, NH₂, N(H)—C₁₋₃ alkyl, N(C₁₋₃ alkyl)₂, C₃₋₆ cycloalkyl, AryA, HetA, or HetR, or
      (5) C₃₋₆ cycloalkyl, AryA, HetA, or HetR, or
   (b) C(O)OR^{D}, wherein R^{D} is:
      (1) C₁₋₅ alkyl,
      (2) O—C₁₋₅ alkyl, or
      (3) C₁₋₅ alkyl substituted with OH, O—C₁₋₅ alkyl, AryA, HetA, or HetR,

then R¹ is not:
(1) C₁₋₈ alkyl,
(2) C₁₋₅ alkyl substituted with OH or O—C₁₋₅ alkyl, or
(3) HetS.

The present invention also includes compounds of Formula II and pharmaceutically acceptable salts thereof:

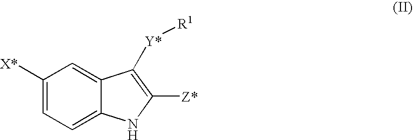

wherein:
X* is halogen;

### Y* is S, S(O), or S(O)₂;

R³ is C₁₋₆ alkyl;
Z* is C(O)NHR⁴, wherein R⁴ is C₁₋₆ alkyl substituted with HetQ; and HetQ is:
(i) a 5-membered heteroaromatic ring containing from 1 to 3 heteroatoms independently selected from 1 to 3 N atoms, from zero to 1 O atom, and from zero to 1 S atom, wherein the heteroaromatic ring is optionally substituted with from 1 to 2 substituents each of which is independently:
(1) C₁₋₆ alkyl,
(2) C₁₋₆ alkyl substituted with OH or O—C₁₋₆ alkyl,
(3) O—C₁₋₁₆ alkyl,
(4) C₁₋₆ haloalkyl,
(5) O—C₁₋₆ haloalkyl,
(6) OH,
(7) Cl, Br, or F,
(8) CN,
(9) C(O)N(H)—C₁₋₆ alkyl,
(10) C(O)N(C₁₋₆ alkyl)₂,
(11) S(O)₂—C₁₋₁₆ alkyl,
(12) S(O)₂NH₂,
(13) S(O)₂N(H)—C₁₋₆ alkyl,
(14) S(O)₂N(C₁₋₁₆ alkyl)₂,
(15) C₃₋₆ cycloalkyl which is optionally substituted with C₁₋₆ alkyl or phenyl,
(16) phenyl which is optionally substituted with from 1 to 3 substituents each of which is independently C₁₋₆ alkyl, O—C₁₋₆ alkyl, C₁₋₆ haloalkyl, O—C₁₋₆ haloalkyl, OH, halogen, NO₂, C(O)N(H)C₁₋₆ alkyl, C(O)N(C₁₋₆ alkyl)₂, CO₂—C₁₋₁₆ alkyl, or S(O)₂—C₁₋₆ alkyl,
(17) phenyl substituted with a saturated heterocyclic ring selected from the group consisting of

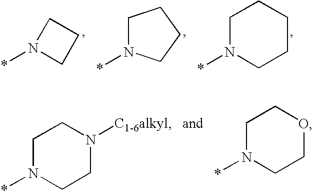

wherein the asterisk denotes the point of attachment to the rest of the molecule,
(18) C₁₋₆ alkyl substituted with C₃₋₆ cycloalkyl, or
(19) C₁₋₆ alkyl substituted with phenyl or O-phenyl, wherein the phenyl is optionally substituted with from 1 to 3 substituents each of which is independently C₁₋₆ alkyl, O—C₁₋₆ alkyl, C₁₋₆ haloalkyl, O—C₁₋₁₆ haloalkyl, OH, halogen, NO₂, C(O)N(H)C₁₋₆ alkyl, C(O)N(C₁₋₆ alkyl)₂, CO₂—C₁₋₆ alkyl, or S(O)₂—C₁₋₆ alkyl, or
(ii) a 5-membered heteroaromatic ring containing from 1 to 2 heteroatoms independently selected from 1 to 2 N atoms, from zero to 1 O atom, and from zero to 1 S atom, wherein the heteroaromatic ring has fused thereto a 6-membered carbocyclic ring that is saturated or partially or fully unsaturated, wherein the fused ring system is optionally substituted with from 1 to 4 substituents each of which is independently
(1) C₁₋₆ alkyl,
(2) C₁₋₆ alkyl substituted with OH or O—C₁₋₆ alkyl,
(3) O—C₁₋₆ alkyl,
(4) C₁₋₆ haloalkyl,
(5) O—C₁₋₆ haloalkyl,
(6) OH,
(7) Cl, Br, or F,
(8) CN,
(9) C(O)N(H)—C₁₋₆ alkyl,
(10) C(O)N(C₁₋₆ alkyl)₂,
(11) S(O)₂—C₁₋₆ alkyl,
(12) S(O)₂NH₂,
(13) S(O)₂N(H)—C₁₋₆ alkyl, or
(14) S(O)₂N(C₁₋₆ alkyl)₂.
(15) C₃₋₆ cycloalkyl which is optionally substituted with C₁₋₆ alkyl or phenyl,
(16) phenyl which is optionally substituted with from 1 to 3 substituents each of which is independently C₁₋₆ alkyl, O—C₁₋₆ alkyl, C₁₋₆ haloalkyl, O—C₁₋₆ haloalkyl, OH, halogen, NO₂, C(O)N(H)C₁₋₆ alkyl, C(O)N(C₁₋₆ alkyl)₂, CO₂—C₁₋₆ alkyl, or S(O)₂—C₁₋₆ alkyl,
(17) phenyl substituted with a saturated heterocyclic ring selected from the group consisting of:

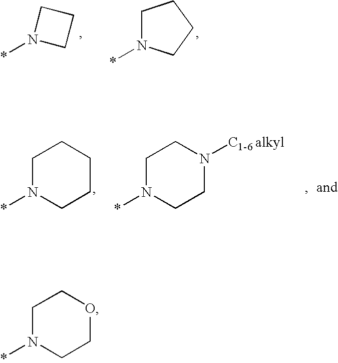

wherein the asterisk denotes the point of attachment to the rest of the molecule,
(18) C₁₋₆ alkyl substituted with C₃₋₆ cycloalkyl, or
(19) C₁₋₆ alkyl substituted with phenyl or O-phenyl, wherein the phenyl is optionally substituted with from 1 to 3 substituents each of which is independently C₁₋₆ alkyl, O—C₁₋₆ alkyl, C₁₋₆ haloalkyl, O—C₁₋₆ haloalkyl, OH, halogen, NO₂, C(O)N(H)C₁₋₆ alkyl, C(O)N(C₁₋₆ alkyl)₂, CO₂—C₁₋₁₆ alkyl, or S(O)₂—C₁₋₁₆ alkyl.

Embodiments, aspects and features of the present invention are either further described in or will be apparent from the ensuing description, examples and appended claims.

### DETAILED DESCRIPTION OF THE INVENTION

The compounds of Formulas I and II above, and pharmaceutically acceptable salts thereof, are HIV reverse transcriptase inhibitors. The compounds are useful for inhibiting HIV reverse transcriptase and for inhibiting HIV replication in vitro and in vivo. More particularly, the compounds of Formula I inhibit the polymerase function of HIV-1 reverse transcriptase. Based upon the testing of representative compounds of the invention in the assay set forth in Example 42 below, it is known that compounds of Formula I inhibit the RNA-dependent DNA polymerase activity of HIV-1 reverse transcriptase. Certain compounds of the present invention can also exhibit activity against drug resistant forms of HIV (e.g., mutant strains of HIV in which reverse transcriptase has a mutation at lysine 103 asparagine (K103N) and/or tyrosine 181→cysteine (Y181C)), and thus can exhibit decreased cross-resistance against currently approved antiviral therapies.

A first embodiment of the present invention is a compound of Formula I, or a pharmaceutically acceptable salt thereof, wherein each of the variables is as originally defined above (i.e., as defined in the Summary of the Invention); and with the proviso that:

(A) when:
(i) X is halogen, NO₂, CN, OH, O—C₁₋₆ alkyl, N(R^{A})R^{B}, N(R^{A})SO₂R^{B}, or N(R^{A})C(O)R^{B},
(ii) R² is H, and
(iii) Z is:
   (a) C(O)N(H)R^{C}, wherein R^{C} is:
      (1) H,
      (2) C₁₋₆ alkyl,
      (3) O—C₁₋₆ alkyl,
      (4) C₁₋₆ alkyl substituted with OH, O—C₁₋₆ alkyl, OC(O)R^{A}, CO₂R^{A}, N(R^{A})R^{B}, CycA, AryA, HetA, or HetR, or
      (5) CycA, AryA, HetA, or HetR, or
   (b) C(O)OR^{D}, wherein R^{D} is:
      (1) C₁₋₆ alkyl,
      (2) O—C₁₋₆ alkyl, or
      (3) C₁₋₆ alkyl substituted with OH, O—C₁₋₆ alkyl, AryA, HetA, or HetR,

then R¹ is not:
(1) C₁₋₈ alkyl,
(2) C₁₋₈ alkyl substituted with OH or O—C₁₋₆ alkyl, or
(3) HetS.

A second embodiment of the present invention is a compound of Formula I, or a pharmaceutically acceptable salt thereof, wherein X is halogen; and all other variables are as originally defined.

A third embodiment of the present invention is a compound of Formula I, or a pharmaceutically acceptable salt thereof, wherein X is chloro or bromo; and all other variables are as originally defined.

A fourth embodiment of the present invention is a compound of Formula I, or a pharmaceutically acceptable salt thereof, wherein X is chloro; and all other variables are as originally defined.

A fifth embodiment of the present invention is a compound of Formula I, or a pharmaceutically acceptable salt thereof, wherein Y is S or S(O)₂; and all other variables are as originally defined or as defined in any of the preceding embodiments of Formula I.

A sixth embodiment of the present invention is a compound of Formula I, or a pharmaceutically acceptable salt thereof, wherein Y is S; and all other variables are as originally defined or as defined in any of the preceding embodiments of Formula I.

A seventh embodiment of the present invention is a compound of Formula I, or a pharmaceutically acceptable salt thereof, wherein Y is S(O)₂; and all other variables are as originally defined or as defined in any of the preceding embodiments of Formula I.

An eighth embodiment of the present invention is a compound of Formula I, or a pharmaceutically acceptable salt thereof, wherein Z is C(O)NH₂, C(O)NH—C₁₋₆ alkyl, or C(O)O—C₁₋₆ alkyl; and all other variables are as originally defined or as defined in any of the preceding embodiments of Formula I.

A ninth embodiment of the present invention is a compound of Formula I, or a pharmaceutically acceptable salt thereof, wherein Z is C(O)NH₂, C(O)NH—C₁₋₄ alkyl, or C(O)O—C₁₋₄ alkyl; and all other variables are as originally defined or as defined in any of the preceding embodiments of Formula I.

A tenth embodiment of the present invention is a compound of Formula I, or a pharmaceutically acceptable salt thereof, wherein Z is C(O)NH₂, C(O)N(M)CH₃, C(O)N(H)CH₂CH₃, C(O)OCH₃, or C(O)OCH₂CH₃; and all other variables are as originally defined or as defined in any of the preceding embodiments of Formula I.

An eleventh embodiment of the present invention is a compound of Formula I, or a pharmaceutically acceptable salt thereof, wherein Z is C(O)NH₂; and all other variables are as originally defined or as defined in any of the preceding embodiments of Formula I.

A twelfth embodiment of the present invention is a compound of Formula I, or a pharmaceutically acceptable salt thereof, wherein R¹ is: (1) C₁₋₆ haloalkyl, (2) CycB, or (3) C₁₋₆ alkyl substituted with CycB; and all other variables are as originally defined or as defined in any of the preceding embodiments of Formula I.

A thirteenth embodiment of the present invention is a compound of Formula I, or a pharmaceutically acceptable salt thereof, wherein R¹ is: (1) C₁₋₆ fluoroalkyl, (2) CycB, or (3) CH₂-CycB, CH₂CH₂-CycB, or CH(CH₃)-CycB; and all other variables are as originally defined or as defined in any of the preceding embodiments of Formula I.

A fourteenth embodiment of the present invention is a compound of Formula I, or a pharmaceutically acceptable salt thereof, wherein R¹ is: (1) CH₂CF₃, (2) CH₂CH₂CF₃, (3) CH₂CF₂CF₃, (4) CH₂CF₂CHF₂, (5) CH₂CH₂CH₂CF₃, (6) CH(CH₃)CH₂CF₃, (7) CH(CF₃)CH₂CF₃, (8) CH(CF₃)₂, (9) CycB, (10) CH₂-CycB, (11) CH₂CH₂-CycB, or (12) CH(CH₃)-CycB; and all other variables are as originally defined or as defined in any of the preceding embodiments of Formula I.

A fifteenth embodiment of the present invention is a compound of Formula I, or a pharmaceutically acceptable salt thereof, wherein CycB is C₃₋₆ cycloalkyl or C₅₋₆ cycloalkenyl, wherein the cycloalkyl or cycloalkenyl is optionally substituted with a total of from 1 to 4 substituents, each of which is independently Cl, Br, F, C₁₋₄ alkyl, O—C₁₋₄ alkyl, CF₃, or C(O)O—C₁₋₄ alkyl; and all other variables are as originally defined or as defined in any of the preceding embodiments of Formula I.

A fifteenth embodiment of the present invention is a compound of Formula I, or a pharmaceutically acceptable salt thereof, wherein CycB is cyclopropyl, cyclobutyl, cyclopentyl, cyclohexyl, cyclohexenyl,

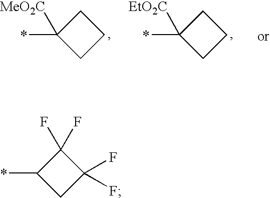

and all other variables are as originally defined or as defined in any of the preceding embodiments of Formula I.

As may be seen by reference to the fifteenth embodiment, a substituent on the cycloalkyl or cycloalkenyl group defined in CycB can be on the ring carbon attached to the rest of the molecule, provided a stable molecule results. Accordingly, a sixteenth embodiment of the present invention is a compound of Formula I, or a pharmaceutically acceptable salt thereof, wherein CycB is:

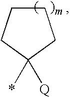

wherein the asterisk * denotes the point of attachment to the rest of the molecule; m is an integer equal to zero, 1, or 2; Q is C(O)OR^{A} or C₁₋₆ alkyl; and all other variables are as originally defined or as defined in any of the preceding embodiments of Formula I.

A seventeenth embodiment of the present invention is a compound of Formula I, or a pharmaceutically acceptable salt thereof, wherein R² is H; and all other variables are as originally defined or as defined in any of the preceding embodiments of Formula I.

It is understood that, to the extent it is applicable, proviso A set forth in the original definition of compounds of Formula I is included in the definition of the second to seventeenth embodiments and that, when the proviso is applied, the proviso can be modified to conform with the definitions of the variables set forth in the particular embodiment. It is also understood that, to the extent it is applicable, proviso A as set forth in the first embodiment is included as an aspect of each of the second to seventeenth embodiments and that, when the proviso is applied, it can be modified to conform with the definitions of the variables set forth in the particular embodiment.

A first class of the present invention includes compounds of Formula I and pharmaceutically acceptable salts thereof, wherein:

X is halogen;

Y is S, S(O), or S(O)₂;

Z is C(O)NH₂, C(O)NH—C₁₋₆ alkyl, or C(O)O—C₁₋₆ alkyl;

R¹ is:
(1) C₁₋₆ haloalkyl,
(2) CycB, or
(3) C₁₋₆ alkyl substituted with CycB; and

R² is H.

A second class of the present invention includes compounds of Formula I-A and pharmaceutically acceptable salts thereof:

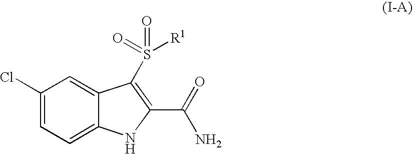

wherein:

R¹ is:
(1) C₁₋₆ fluoroalkyl,
(2) CycB, or
(3) CH₂-CycB, CH₂CH₂-CycB, or CH(CH₃)-CycB; and

CycB is C₃₋₆ cycloalkyl or C₅₋₆ cycloalkenyl, wherein the cycloalkyl or cycloalkenyl is optionally substituted with a total of from 1 to 4 substituents, each of which is independently Cl, Br, F, C₁₋₄ alkyl, O—C₁₋₄ alkyl, CF₃, or C(O)O—C₁₋₄ alkyl.

A third class of the present invention includes compounds of Formula I-B and pharmaceutically acceptable salts thereof:

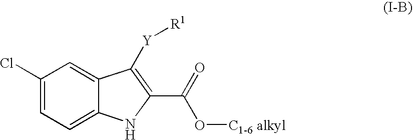

wherein:

Y is S or S(O)₂;

R¹ is:
(1) C₁₋₆ fluoroalkyl,
(2) CycB, or
(3) CH₂-CycB, CH₂CH₂-CycB, or CH(CH₃)-CycB; and

CycB is C₃₋₆ cycloallyl or C₅₋₆ cycloalkenyl, wherein the cycloalkyl or cycloalkenyl is optionally substituted with a total of from 1 to 4 substituents, each of which is independently Cl, Br, F, C₁₋₄ alkyl, O—C₁₋₄ alkyl, CF₃, or C(O)O—C₁₋₄ alkyl.

An eighteenth embodiment of the present invention is a compound of Formula II, or a pharmaceutically acceptable salt thereof, wherein X* is chloro or bromo; and all other variables are as originally defined.

A nineteenth embodiment of the present invention is a compound of Formula II, or a pharmaceutically acceptable salt thereof, wherein X* is chloro; and all other variables are as originally defined.

A twentieth embodiment of the present invention is a compound of Formula II, or a pharmaceutically acceptable salt thereof, wherein X* is bromo; and all other variables are as originally defined.

A twenty-first embodiment of the present invention is a compound of Formula II, or a pharmaceutically acceptable salt thereof, wherein Y* is S or S(O)₂; and all other variables are as originally defined or as defined in any of the preceding embodiments of Formula II.

A twenty-second embodiment of the present invention is a compound of Formula II, or a pharmaceutically acceptable salt thereof, wherein Y* is S; and all other variables are as originally defined or as defined in any of the preceding embodiments of Formula II.

A twenty-third embodiment of the present invention is a compound of Formula II, or a pharmaceutically acceptable salt thereof, wherein Y* is S(O)₂; and all other variables are as originally defined or as defined in any of the preceding embodiments of Formula II.

A twenty-fourth embodiment of the present invention is a compound of Formula II, or a pharmaceutically acceptable salt thereof, wherein R³ is C₁₋₅ alkyl; and all other variables are as originally defined or as defined in any of the preceding embodiments of Formula II.

A twenty-fifth embodiment of the present invention is a compound of Formula II, or a pharmaceutically acceptable salt thereof, wherein R⁴ is (CH₂)₁₋₃-HetQ or CH(CH₃)-HetQ; and all other variables are as originally defined or as defined in any of the preceding embodiments of Formula II. An aspect of this embodiment is a compound of Formula II, or a pharmaceutically acceptable salt thereof, wherein R⁴ is (CH₂)₁₋₃-HetQ. Other aspects of this embodiment include a compound of Formula II in which R⁴ is (CH₂)₁₋₂-HetQ; R⁴ is (CH₂)₂-HetQ; and R⁴ is CH(CH₃)-HetQ.

A twenty-sixth embodiment of the present invention is a compound of Formula II, or a pharmaceutically acceptable salt thereof, wherein R⁴ is CH₂-HetQ; and all other variables are as originally defined or as defined in any of the preceding embodiments of Formula II.

A twenty-seventh embodiment of the present invention is a compound of Formula II, or a pharmaceutically acceptable salt thereof, wherein HetQ is:

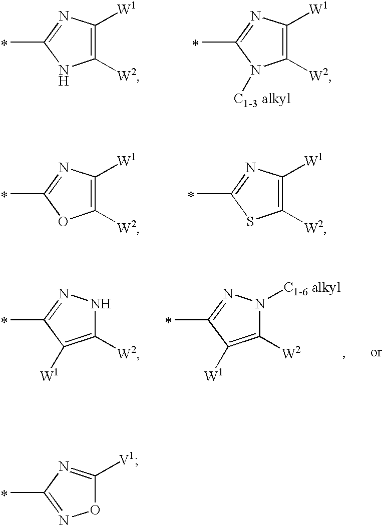

### V¹ is:

(1) H,
(2) C₁₋₃ alkyl,
(3) C₁₋₃ alkyl substituted with OH or OCH₃,
(4) C₃₋₆ cycloalkyl which is optionally substituted with C₁₋₄ alkyl or phenyl,
(5) phenyl which is optionally substituted with from 1 to 3 substituents each of which is independently CH₃, OCH₃, CF₃, OCF₃, OH, Cl, Br, F, CN, NO₂, C(O)N(H)CH₃, C(O)N(CH₃)₂, CO₂CH₃, or S(O)₂CH₃,
(6) phenyl substituted with a saturated heterocyclic ring selected from the group consisting

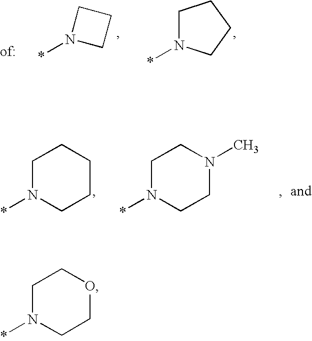

wherein the asterisk denotes the point of attachment to the rest of the molecule,
(7) CH₂-cyclopropyl,
(8) CH₂-phenyl, or
(9) CH₂—O-phenyl;
   W¹ and W² each independently have the same definition as V¹;
   or alternatively, W¹ and W² together with the carbon atoms to which each is attached form a benzo ring; and all other variables are as originally defined or as defined in any of the preceding embodiments of Formula II.

A twenty-eighth embodiment of the present invention is a compound of Formula II, or a pharmaceutically acceptable salt thereof, wherein HetQ is:

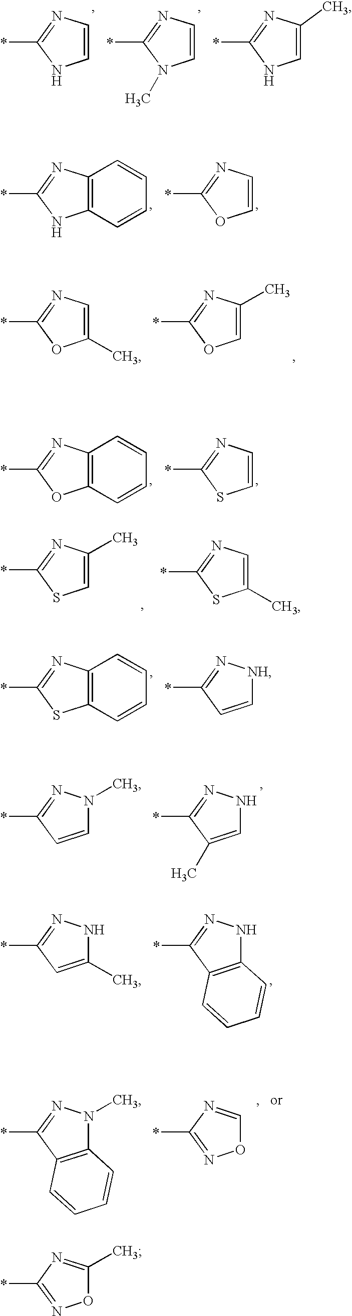

and all other variables are as originally defined or as defined in any of the preceding embodiments of Formula II.

A fourth class of the present invention includes compounds of Formula II and pharmaceutically acceptable salts thereof, wherein: X* is chloro or bromo; Y* is S or S(O)₂; R³ is C₁₋₅ alkyl; R⁴ is (CH₂)₁₋₃-HetQ or CH(CH₃)-HetQ; and HetQ is as defined in the twenty-seventh embodiment.

A fifth class of the present invention includes compounds of Formula II and pharmaceutically acceptable salts thereof, wherein: X* is chloro; Y* is S(O)₂; R³ is C₁₋₅ alkyl; R⁴ is CH₂-HetQ; and HetQ is as defined in the twenty-eighth embodiment.

Another embodiment of the present invention is a compound, or a pharmaceutically acceptable salt thereof, selected from the group consisting of the compounds set forth in Examples 1 to 40 below. In an aspect of this embodiment, the compound is selected from the group consisting of the compounds set forth in Examples 1 to 38. In another aspect of this embodiment, the compound is selected from the group consisting of the compounds set forth in Examples 39 to 40.

Another embodiment of the present invention is a compound of Formula I, or a pharmaceutically acceptable salt thereof, as originally defined or as defined in any of the foregoing embodiments, classes, aspects, or features of Formula I, wherein the compound or its salt is substantially pure. Still another embodiment of the present invention is a compound of Formula I, or a pharmaceutically acceptable salt thereof, as originally defined or as defined in any of the foregoing embodiments, classes, aspects, or features of Formula I, wherein the compound or its salt is substantially pure. As used herein “substantially pure” means that the compound or its salt is present (e.g., in a product isolated from a chemical reaction or a metabolic process) in an amount of at least about 90 wt. % (e.g., from about 95 wt. % to 100 wt. %), preferably at least about 95 wt. % (e.g., from about 98 wt. % to 100 wt. %), more preferably at least about 99 wt. %, and most preferably 100 wt. %. The level of purity of the compounds and salts can be determined using standard methods of analysis. A compound or salt of 100% purity can alternatively be described as one which is free of detectable impurities as determined by one or more standard methods of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

Other embodiments of the present invention include the following:

(a) A pharmaceutical composition comprising an effective amount of Compound I as originally defined above (including proviso A), or a pharmaceutically acceptable salt thereof, and a pharmaceutically acceptable carrier.

(b) A pharmaceutical composition which comprises the product prepared by combining (e.g., mixing) an effective amount of Compound I as originally defined above (including proviso A), or a pharmaceutically acceptable salt thereof, and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, HIV reverse transcriptase inhibitors other than a compound of Formula I, and HIV integrase inhibitors.

(e) A pharmaceutical combination which is (i) a compound of Formula I as originally defined above (including proviso A), or a pharmaceutically acceptable salt thereof, and (ii) an anti-HIV agent selected from the group consisting of HIV antiviral agents, immunomodulators, and anti-infective agents; wherein the compound of Formula I and the anti-HIV agent are each employed in an amount that renders the combination effective for inhibition of HIV reverse transcriptase, for treatment or prophylaxis of infection by HIV, or for treatment, prophylaxis of, or delay in the onset of AIDS.

(f) The combination of (e), wherein the anti-HIV agent is an antiviral selected from the group consisting of HIV protease inhibitors, HIV reverse transcriptase inhibitors other than a compound of Formula I, and HIV integrase inhibitors.

Additional embodiments of the invention include the pharmaceutical compositions and combinations set forth in (a)-(f) above, wherein the compound of the present invention employed therein is a compound defined in one of the embodiments, classes, or aspects of Formula I described above, wherein it is understood that the definitions include the accompanying proviso. In all of these embodiments, the compound can optionally be used in the form of a pharmaceutically acceptable salt.

Additional embodiments of the present invention include each of the pharmaceutical compositions and combinations set forth in (a)-(f) above and embodiments thereof, wherein the compound of the present invention or its salt employed therein is substantially pure. With respect to a pharmaceutical composition comprising a compound of Formula I or its salt and a pharmaceutically acceptable carrier and optionally one or more excipients, it is understood that the term “substantially pure” is in reference to Compound I or its salt per se; i.e., the purity of the active ingredient in the composition.

The present invention also includes a method for inhibition of HIV reverse transcriptase, for treatment or prophylaxis of HIV infection, or for treatment, prophylaxis of, or delay in the onset of AIDS, which comprises administering to a subject in need thereof an effective amount of a compound of Formula I, or a pharmaceutically acceptable salt thereof, wherein Formula I is as originally set forth and defined above (including proviso A). Embodiments of the method of the present invention include those in which the compound of Formula I administered to the subject is as defined in the compound embodiments, classes and aspects of Formula I set forth above.

The present invention also includes a compound of Formula I, or a pharmaceutically acceptable salt thereof, (i) for use in, (ii) for use as a medicament for, or (iii) for use in the preparation of a medicament for: (a) inhibition of HIV reverse transcriptase, (b) treatment or prophylaxis of infection by HIV, or (c) treatment, prophylaxis of, or delay in the onset of AIDS. In these uses, the compound of Formula I is as originally set forth and defined above, including proviso A (i.e., proviso A is applied). In these uses, the compounds of the present invention can optionally be employed in combination with one or more anti-HIV agents selected from HIV antiviral agents, anti-infective agents, and immunomodulators. Embodiments of the uses of the present invention include those in which the compound of Formula I is as defined in the compound embodiments, classes and aspects set forth above.

Still other embodiments of the present invention include pharmaceutical composition and combination embodiments for Compound II corresponding to embodiments (a)-(f) set forth above for Compound I; i.e., the embodiments for Compound II are identical to those of Compound I except that each occurrence of “Compound I” is replaced with “Compound II”.

The present invention also includes a method for inhibition of HIV reverse transcriptase, for treatment or prophylaxis of HIV infection, or for treatment, prophylaxis of, or delay in the onset of AIDS, which comprises administering to a subject in need thereof an effective amount of a compound of Formula II, or a pharmaceutically acceptable salt thereof, wherein Formula II is as originally set forth and defined above. Embodiments of the method of the present invention include those in which the compound of Formula II administered to the subject is as defined in the compound embodiments, classes and aspects of Formula II set forth above.

The present invention also includes a compound of Formula II, or a pharmaceutically acceptable salt thereof, (i) for use in, (ii) for use as a medicament for, or (iii) for use in the preparation of a medicament for: (a) inhibition of HIV reverse transcriptase, (b) treatment or prophylaxis of infection by HIV, or (c) treatment, prophylaxis of, or delay in the onset of AIDS. In these uses, the compound of Formula II is as originally set forth and defined above. In these uses, the compounds of the present invention can optionally be employed in combination with one or more anti-HIV agents selected from HIV antiviral agents, anti-infective agents, and immunomodulators. Embodiments of the uses of the present invention include those in which the compound of Formula II is as defined in the compound embodiments, classes and aspects set forth above.

As used herein, the term “alkyl” refers to any linear or branched chain alkyl group having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and isopropyl, ethyl and methyl. As another example, “C₁₋₄ alkyl” refers to n-, iso-, seq- and t-butyl, n- and isopropyl, ethyl and methyl.

The term “alkylene” refers to any divalent linear or branched chain aliphatic hydrocarbon radical (or alternatively an “alkanediyl”) having a number of carbon atoms in the specified range. Thus, for example, “—C₁₋₆ alkylene-” refers to any of the C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Another sub-class of interest an alkylene selected from the group consisting of —CH₂—, —CH(CH₃)—, and —C(CH₃)₂—.

The term “cycloalkyl” refers to any cyclic ring of an alkane having a number of carbon atoms in the specified range. Thus, for example, “C₃₋₈ cycloalkyl” (or “C₃-C₈ cycloalkyl”) refers to cyclopropyl, cyclobutyl, cyclopentyl, cyclohexyl, cycloheptyl, and cyclooctyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.). Fluoroalkyls of particular interest include CF₃, CH₂CF₃, CH₂CH₂CF₃, CF₂CF₃, and CH₂CF₂CF₃.

The term “C(O)” appearing in the definition of a functional group (e.g., “C(O)R^{A}”) refers to carbonyl. The term “S(O)₂” or “SO₂” appearing in the definition of a functional group refers to sulfonyl, the term “S(O)” refers to sulfinyl, and the terms “C(O)O” and “CO₂” both refer to carboxyl.

The term “Compound I” refers to a compound of Formula I. The term “Compound II” refers to a compound of Formula II.

The symbol “*” at the end of a bond each refer to the point of attachment of a functional group or other chemical moiety to the rest of the molecule of which it is a part.

Unless expressly stated to the contrary in a particular context, any of the various carbocyclic and heterocyclic rings and ring systems defined herein may be attached to the rest of the compound at any ring atom (i.e., any carbon atom or any heteroatom) provided that a stable compound results. Suitable aryls include phenyl, 9- and 10-membered bicyclic, fused carbocyclic ring systems, and 11- to 14-membered tricyclic fused carbocyclic ring systems, wherein in the fused carbocyclic ring systems at least one ring is aromatic. Suitable aryls include, for example, phenyl, naphthyl, tetrahydronaphthyl (tetralinyl), indenyl, anthracenyl, and fluorenyl. Suitable heteroaryls include 5- and 6-membered heteroaromatic rings and 9- and 10-membered bicyclic, fused ring systems in which at least one ring is aromatic, wherein the heteroaromatic ring or the bicyclic, fused ring system contains from 1 to 4 heteroatoms independently selected from N, O and S, wherein each N is optionally in the form of an oxide and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl. Suitable heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl (e.g., benzo-1,3-dioxolyl:

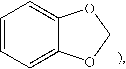

benzopiperidinyl, benzisoxazolyl, benzoxazolyl, chromanyl, isochromanyl, benzothienyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, and 2,3-dihydrobenzo-1,4-dioxinyl (i.e.,

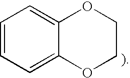

Suitable saturated and mono-unsaturated heterocyclic rings include 4- to 7-membered saturated and mono-unsaturated heterocyclic rings containing at least one carbon atom and from 1 to 4 heteroatoms independently selected from N, O and S, wherein each S is optionally oxidized to S(O) or S(O)₂. Suitable 4- to 7-membered saturated heterocyclics include, for example, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Suitable mono-unsaturated heterocyclic rings include those corresponding to the saturated heterocyclic rings listed in the preceding sentence in which a single bond is replaced with a double bond (e.g., a carbon-carbon single bond is replaced with a carbon-carbon double bond). It is understood that the specific rings and ring systems suitable for use in the present invention are not limited to those listed in this paragraph. The rings and ring systems listed in this paragraph are merely representative.

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 to 4 heteroatoms, 1 to 3 heteroatoms, 2 to 3 heteroatoms, 1 to 2 heteroatoms, 1 heteroatom, 2 heteroatoms, 3 heteroatoms, and 4 heteroatoms. As another example, an aryl or heteroaryl described as optionally substituted with “from 1 to 5 substituents” is intended to include as aspects thereof, an aryl or heteroaryl optionally substituted with 1 to 4 substituents, 1 to 3 substituents, 1 to 2 substituents, 2 to 5 substituents, 2 to 4 substituents, 2 to 3 substituents, 3 to 5 substituents, 3 to 4 substituents, 4 to 5 substituents, 1 substituent, 2 substituents, 3 substituents, 4 substituents, and 5 substituents.

When any variable (e.g., R^{A}, R^{B}, AryE, or HetE) occurs more than one time in any constituent or in Formula I or in any other formula depicting and describing compounds employed in the invention, its definition on each occurrence is independent of its definition at every other occurrence. Also, combinations of substituents and/or variables are permissible only if such combinations result in stable compounds.

The term “substituted” (e.g., as in “is optionally substituted with from 1 to 5 substituents . . . ”) includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., cycloalkyl, aryl, or heteroaryl) provided such ring substitution is chemically allowed and results in a stable compound. Ring substituents can be attached to the ring atom which is attached to the rest of the molecule; e.g., methyl-substituted 3-oxetanyl refers to:

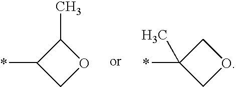

As a result of the selection of substituents and substituent patterns, certain compounds of the present invention can exhibit keto-enol tautomerism. All tautomeric forms of these compounds, whether individually or in mixtures, are within the scope of the present invention. For example, in instances where a hydroxy (—OH) substituent(s) is (are) permitted on a heteroaromatic ring and keto-enol tautomerism is possible, it is understood that the substituent might in fact be present, in whole or in part, in the keto form, as exemplified here for a hydroxypyridinyl substituent:

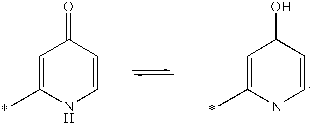

Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain compounds of the present invention can have asymmetric centers and can occur as mixtures of stereoisomers, or as individual diastereomers, or enantiomers. All isomeric forms of these compounds, whether individually or in mixtures, are within the scope of the present invention.

The method of the present invention involves the use of compounds of the present invention in the inhibition of HIV reverse transcriptase (wild type and/or mutant strains thereof), the prophylaxis or treatment of infection by human immunodeficiency virus (HIV) and the prophylaxis, treatment or delay in the onset of consequent pathological conditions such as AIDS. Prophylaxis of AIDS, treating AIDS, delaying the onset of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery. As another example, the present invention can also be employed to prevent transmission of HIV from a pregnant female infected with HIV to her unborn child or from an HIV-infected female who is nursing (i.e., breast feeding) a child to the child via administration of an effective amount of a compound of Formula I or Formula II, or a pharmaceutically acceptable salt thereof.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Certain of the compounds employed in the present invention carry an acidic moiety (e.g., —COOH or a phenolic group), in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of Formula I mean providing the compound or a prodrug of the compound to the individual in need of treatment or prophylaxis. When a compound or a prodrug thereof is provided in combination with one or more other active agents (e.g., antiviral agents useful for treating or prophylaxis of HIV infection or AIDS), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV reverse transcriptase (wild type and/or mutant strains thereof) and thereby elicit the response being sought (i.e., an “inhibition effective amount”). When the active compound (i.e., active ingredient) is administered as the salt, references to the amount of active ingredient are to the free form (i.e., the non-salt form) of the compound.

In the method of the present invention (i.e., inhibiting HIV reverse transcriptase, treating or prophylaxis of HIV infection or treating, prophylaxis of, or delaying the onset of AIDS), the compounds of Formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

The compounds of the present invention (i.e., compounds of Formula I and II) can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to the use of the compounds of the present invention in combination with one or more agents useful in the treatment of HIV infection or AIDS. For example, the compounds of Formula I can be effectively administered, whether at periods of pre-exposure and/or post-exposure, in combination with effective amounts of one or more HIV antiviral agents, immunomodulators, antiinfectives, or vaccines useful for treating HIV infection or AIDS, such as those disclosed in Table 1 of WO 01/38332 or in the Table in WO 02/30930. Suitable HIV antiviral agents for use in combination with the compounds of Formula I or Formula II include, for example, HIV protease inhibitors (e.g., indinavir, atazanavir, lopinavir optionally with ritonavir, saquinavir, or nelfinavir), nucleoside HIV reverse transcriptase inhibitors (e.g., abacavir, lamivudine (3TC), zidovudine (AZT), or tenofovir), non-nucleoside HIV reverse transcriptase inhibitors (e.g., efavirenz or nevirapine), and HIV integrase inhibitors such as those described in WO 02/30930, WO 03/35076, and WO 03/35077. It will be understood that the scope of combinations of compounds of Formula I or Formula II with HIV antiviral agents, immunomodulators, anti-infectives or vaccines is not limited to the foreogoing substances or to the list in the above-referenced Tables in WO 01/38332 and WO 02/30930, but includes in principle any combination with any pharmaceutical composition useful for the treatment of HIV infection or AIDS. The HIV antiviral agents and other agents will typically be employed in these combinations in their conventional dosage ranges and regimens as reported in the art, including, for example, the dosages described in the Physicians' Desk Reference, 58^{th} edition, Thomson P D R, 2004. The dosage ranges for a compound of Formula I in these combinations are the same as those set forth above. It is understood that pharmaceutically acceptable salts of the compounds of the invention and/or the other agents (e.g., indinavir sulfate) can be used as well.

Abbreviations employed herein include the following:
dGTP=deoxyguanosine triphosphate
DMF=N,N-dimethylformamide
DMSO=dimethylsufoxide
dNTP=deoxynucleoside triphosphate
EDTA=ethylenediaminetetracetic acid
EGTA=ethylene glycol bis(2-aminoethyl ether)-N,N,N′,N′-tetraacetic acid
Et=ethyl
HPLC=high performance liquid chromatography
m-CPBA=meta-chloroperbenzoic acid
Me=methyl
MeOH=methanol
MS=mass spectroscopy
NBS=N-bromosuccinimide
nnRTI=non-nucleoside reverse transcriptase inhibitor
THF=tetrahydrofuran

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

Scheme 1 provides a method for preparing compounds of the present invention which is similar to the procedure described in Salituro et al., J. Med. Chem. 1992, 35: 1791-1799. In Scheme 1 indole-2-carboxylate 1 can be selectively brominated at C-3 to give the 3-bromoindole 2. Reaction of 2 with a thiol under basic conditions (e.g., potassium carbonate in acetone) and heating either conventionally or with microwaves, gives the sulfide 3. Aminolysis of the ester group on the sulfide on affords 4. Alternatively, 3 can be hydrolyzed with aqueous base to the carboxylic acid, which can then be coupled with a suitable amine to provide amide 4. Oxidation of 4 with a suitable oxidizing agent (e.g., m-CPBA) can give the sulfoxide 5a or the sulfone 5b, depending on the number of equivalents of oxidant used.

Many indole-2-carboxylates encompassed by formula I are commercially available. Alternatively, indole-2-carboxylates of formula I can be prepared via methods known in the art including those described in Joule et al., Science of Synthesis 2001, vol. 10, pp. 361-652.

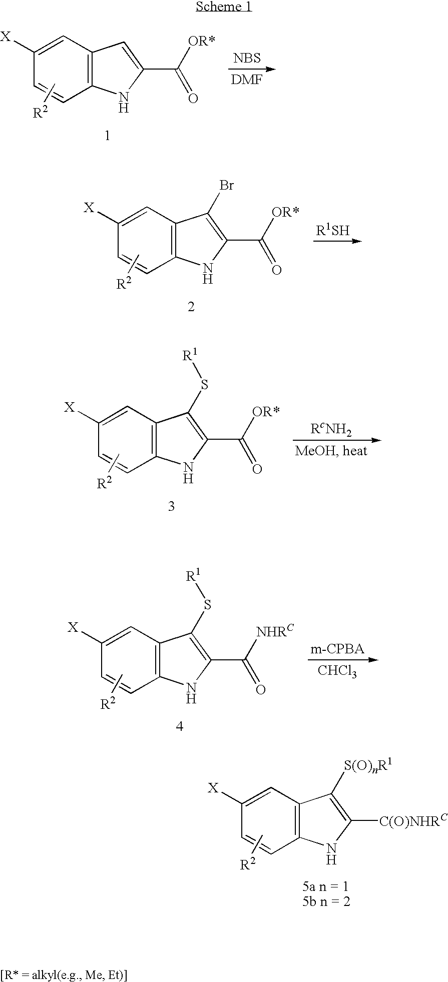

Scheme 2 is a variation on Scheme 1, wherein sulfide 3 is oxidized to give alkyl 3-sulfinyl-indole-2-carboxylate 6a and alkyl 3-sulfonyl-indole-2-carboxylate 6b, each of which can be converted to the corresponding amides 5a and 5b as before.

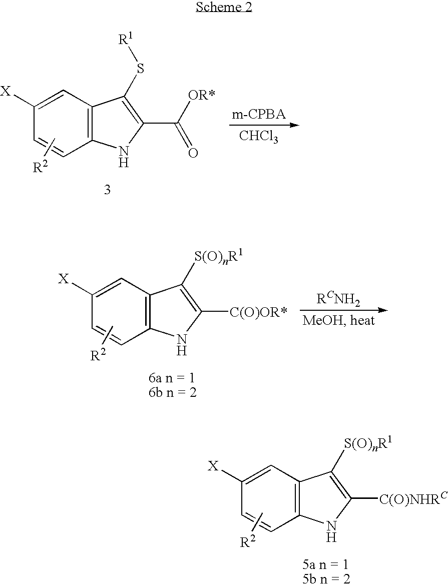

A disadvantage of the synthetic route set forth in Scheme 1 is the use of low molecular weight thiol nucleophiles, which are not readily available commercially and which possess unpleasant odors. Published syntheses of 3-arylthioindoles and 3-arylsulfonylindoles either failed due to poor reactivity or were not attractive routes due to the lack of commercially available starting materials. These include syntheses described in Williams et al., J. Med. Chem. 1993, vol. 36, pp. 1291-1294; Sun et al., Syn Comm. 1998, vol. 28, pp. 1785-1791; Frye et al., J. Org. Chem. 1992, vol. 57, pp. 697-701; Steensma et al., Tetrahedron Lett. 2001, vol. 42, pp. 2281-2283; Yadav et al., Tetrahedron Lett. 2003, vol. 44, pp. 6055-6058; and Silvestri et al., J. Med. Chem, 2003, vol. 46, pp. 2482-2493.

Scheme 3 provides an alternative procedure to that set forth in Scheme 1 and is based on the preparation of 3-mercaptoindole as described in Blank et al., J. Med. Chem. 1977, 20: 572-576. In Scheme 3,3-bromoindole 2 is reacted with potassium thiocyanate to give compound 7, which can then be reacted with a suitable reducing agent (e.g., sodium borohydride in ethanol) to give the 3-mercaptoindole intermediate 8, which is a easily handled, non-volatile solid (i.e., no odor). The 3-mercaptoindole 8 can be alkylated with electrophiles to give the sulfide 3 (e.g., using R¹-bromide, R¹-iodide, R¹—OMs, R¹-OTs in DMF, DMSO, acetonitrile, or acetone and in the presence of a base such as K₂CO₃, sodium carbonate, or cesium carbonate; or using R¹-bromide, R¹-iodide, R¹—OMs, R¹—OTs in DMF, DMSO, with sodium hydride as base). Oxidation of sulfide 3 in the manner set forth in Scheme 2 provides compounds 6a and 6b, and amination with and without oxidation in the manner set forth in Schemes 1 and 2 provides amides 4, 5a, and 5b.

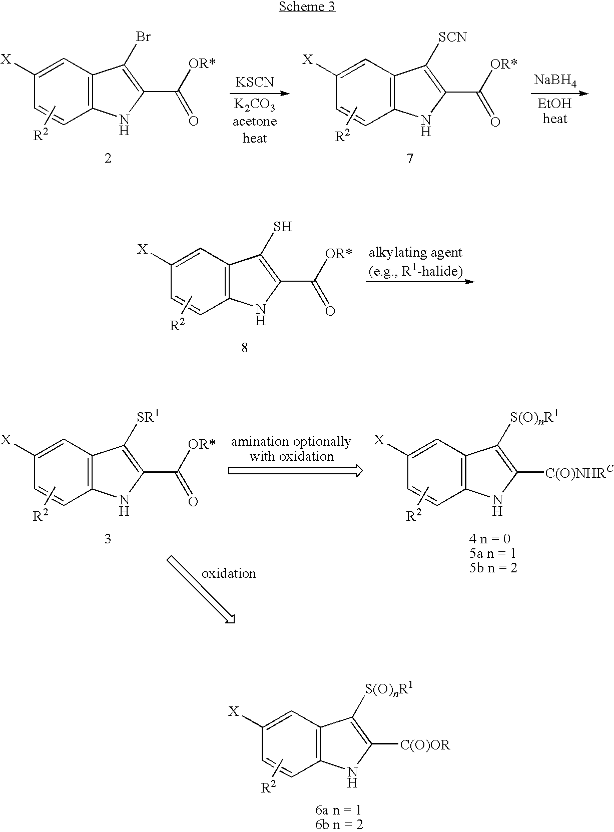

In the processes for preparing compounds of the present invention set forth in the foregoing schemes, functional groups in various moieties and substituents may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2^{nd} edition, 1991. The protecting groups may be removed at a convenient subsequent stage using methods known in the art. Alternatively the interfering group can be introduced into the molecule subsequent to the reaction step of concern.

The following examples serve only to illustrate the invention and its practice. The examples are not to be construed as limitations on the scope or spirit of the invention.

### EXAMPLE 1

### Ethyl 5-chloro-3-(cyclobutylmethylthio)-1H-indole-2-carboxylate

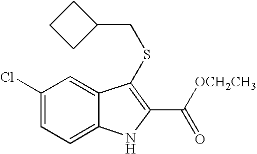

### Step 1: Ethyl 5-chloro-3-thiocyanato-1H-indole-2-carboxylate

A suspension of potassium thiocyanate (6.51 g, 67.0 mmol) in methanol (10 mL) was vigorously stirred and cooled to −78° C. A solution of bromine in methanol (25 mL) was added at such a rate that the temperature did not exceed −60° C. A solution of ethyl 5-chloroindole-2-carboxylate (5.00 g, 22.35 mmol) in methanol (25 mL) at −70° C. was rapidly added in one portion, and the resulting mixture was stirred for 1 hour, and then warmed to room temperature. The reaction mixture was stirred under nitrogen until the reaction was complete. The precipitated solid was washed with methanol and then with water. The product was dried under high vacuum to give the title compound.

### Step 2: Ethyl 5-chloro-3-mercapto-1H-indole-2-carboxylate

Sodium borohydride (0.90 g, 23.9 mmol) was added in portions to a solution of ethyl 5-chloro-3-thiocyanato-1H-indole-2-carboxylate (5.60 g, 19.9 mmol) in ethanol (150 μL). The solution was kept at room temperature for 20 minutes, and then refluxed for 15 minutes. The ethanol was taken off under vacuum, and the residue was suspended in water (200 mL) and stirred for 10 minutes. The solid disulfide by product was filtered and the filtrate was acidified with concentrated hydrochloric acid to pH<4, and then stirred for 5 minutes and filtered. The resulting solid product was then washed with water. The title compound was obtained.

### Step 3: Ethyl 5-chloro-3-(cyclobutylmethylthio)-1H-indole-2-carboxulate

Ethyl 5-chloro-3-mercapto-1H-indole-2-carboxylate (0.100 g, 0.391 mmol), potassium carbonate (65 mg, 0.469 mmol), and (bromomethyl)cyclobutane (0.053 mL, 0.469 mmol) were added to dry DMF (1.5 mL) in a microwave reaction vial. The reaction mixture was purged with nitrogen and heated at 120° C. in the microwave for 20 minutes. After purification by reverse phase chromatography, the title compound was obtained. MS (M+1)=324.1

### EXAMPLES 2-11

The compounds in Table 1 below were prepared using a procedure similar to that employed in Example 1.

TABLE 1

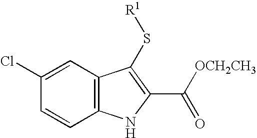

| Ex- | | | |
|---|---|---|---|
| am- | | | MS |
| ple | Name | R¹ | (M + 1) |
| 2 | ethyl 5-chloro-3-[(2,2,2- | CH₂CF₃ | 338.0 |
| | trifluoroethyl)thio]-1H-indole-2- | | |
| | carboxylate | | |
| 3 | ethyl 5-chloro-3-[(3,3,3- | CH₂CH₂CF₃ | 352.1 |
| | trifluoropropyl)thio]-1H-indole-2- | | |
| | carboxylate | | |
| 4 | ethyl 5-chloro-3-[(4,4,4- | CH₂CH₂CH₂CF₃ | 366.05 |
| | trifluorobutyl)thio]-1H-indole-2- | | |
| | carboxylate | | |
| 5 | ethyl 5-chloro-3-[(2,2,3,3,3- | CH₂CF₂CF₃ | 370.1 |
| | pentafluoropropyl)thio]-1H- | | |
| | indole-2-carboxylate | | |
| 6 | ethyl 5-chloro-3- (cyclopropylthio)-1H-indole-2- carboxylate | 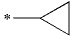 | 296.2 |
| 7 | ethyl 5-chloro-3- [(cyclopropylmethyl)thio]-1H- indole-2-carboxylate | 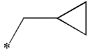 | 342.05 |
| 8 | ethyl 5-chloro-3-[(2- cyclopropylethyl)thio]-1H-indole- 2-carboxylate | 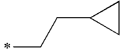 | 296.1 |
| 9 | ethyl 5-chloro-3-(cyclobutylthio)- 1H-indole-2-carboxylate | 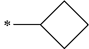 | 310.06 |
| 10 | ethyl 5-chloro-3- (cyclopentylthio)-1H-indole-2- carboxylate | 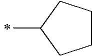 | 324.1 |
| 11 | ethyl 5-chloro-3- [(cyclohexylmethyl)thio]-1H- indole-2-carboxylate | 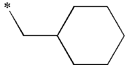 | 352.1 |

### EXAMPLE 12

### Ethyl 5-chloro-3-(cyclohexylthio)-1H-indole-2-carboxylate

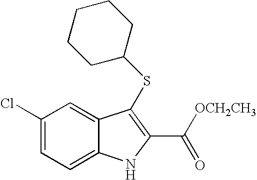

### Step 1: Ethyl 3-bromo-5-chloro-1H-indole-2-carboxylate

A solution of N-bromosuccinimide (0.955 g, 5.36 mmol) in dimethylformamide (10 mL) was slowly added to a solution of ethyl 5-chloroindole-2-carboxylate (1.00 g, 4.47 mmol) in dimethylformamide (25 mL) at 0° C. After 20 minutes, the reaction was poured onto ice (100 mL) and extracted with ether (200 mL). The organic phase was washed with saturated brine, dried over sodium sulfate and concentrated. The crude product was purified by silica get chromatography (eluant: 10% to 30% ethyl acetate in hexane) to give the title compound

### Step 2: Ethyl 5-chloro-3-(cyclohexylthio)-1H-indole-2-carboxylate

Ethyl 3-bromo-5-chloro-1H-indole-2-carboxylate (0.100 g, 0.331 mmol), cyclohexanethiol (0.081 mL, 0.661 mmol, 1.75 eq) and potassium carbonate (91 mg, 0.661 mmol, 1.75 eq.) were combined in acetone (2 ml) in a microwave reaction vial. The reaction was purged with nitrogen and heated in a microwave for 40 minutes at 100° C. Additional cyclohenanethiol was added (1 mL) and heating in the microwave continued for 1 hour at 120° C. The solvent and excess reagent were removed under vacuum and the crude product was dissolved in ethyl acetate, and the organic phase washed with saturated brine. The desired compound was isolated by reverse phase HPLC. MS (M+1)=338.2

### EXAMPLE 13

### Ethyl 5-chloro-3-(cyclobutylmethylsulfonyl)-1H-indole-2-carboxylate

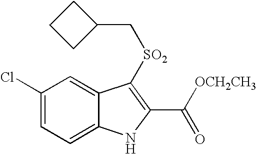

3-mz-Chloroperoxybenzoic acid (59 mg, 0.340 mmol) was added to an ice cooled solution of ethyl 5-chloro-3-(cyclobutylmethylthio)-1H-indole-2-carboxylate (44 mg, 0.135 mmol) in chloroform (2 ml). The reaction mixture was stirred at room temperature overnight. The reaction mixture was then diluted with methylene chloride (10 mL), washed with saturated sodium bicarbonate, dried over sodium sulfate and filtered. The filtrate was concentrated to give the title compound. MS (M+1)=356.07

### EXAMPLES 14-22

The compounds in Table 2 below were prepared using a procedure similar to that employed in Example 13.

TABLE 2

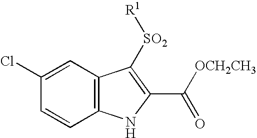

| Ex- | | | |
|---|---|---|---|
| am- | | | MS |
| ple | Name | R¹ | (M + 1) |
| 14 | ethyl 5-chloro-3-[(3,3,3- | CH₂CH₂CF₃ | 384.02 |
| | trifluoropropyl)sulfonyl]-1H- | | |
| | indole-2-carboxylate | | |
| 15 | ethyl 5-chloro-3-[(4,4,4,- | CH₂CH₂CH₂CF₃ | 398.04 |
| | trifluorobutyl)sulfonyl]-1H- | | |
| | indole-2-carboxylate | | |
| 16 | ethyl 5-chloro-3- (cyclopropylsulfonyl)-1H-indole- 2-carboxylate | 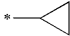 | 328.04 |
| 17 | ethyl 5-chloro-3- [(cyclopropylmethyl)sulfonyl]- 1H-indole-2-carboxylate | 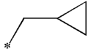 | 342.05 |
| 18 | ethyl 5-chloro-3-[(2- cyclopropylethyl)sulfonyl]-1H- indole-2-carboxylate | 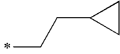 | 356.10 |
| 19 | ethyl 5-chloro-3- (cyclobutylsulfonyl)-1H-indole-2- carboxylate | 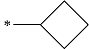 | 342.05 |
| 20 | ethyl 5-chloro-3- (cyclopentylsulfonyl)-1H-indole- 2-carboxylate | 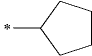 | 356.07 |
| 21 | ethyl 5-chloro-3- (cyclohexylsulfonyl)-1H-indole-2- carboxylate | 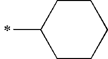 | 370.1 |
| 22 | ethyl 5-chloro-3- [(cyclohexylmethyl)sulfonyl]-1H- indole-2-carboxylate | 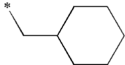 | 384.10 |

### EXAMPLE 23

### 5-Chloro-3-(cyclobutylmethylsulfonyl)-1H-indole-2-carboxamide

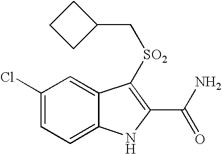

Ethyl 5-chloro-3-(cyclobutylmethylsulfonyl)-1-H-indole-2-carboxylate was dissolved in methanolic ammonia (2 mL, 2M solution) in a pressure vessel. The vessel was sealed and heated to 110° C. until the reaction was complete. The crude product was purified by reverse phase HPLC to give the title compound. MS (M+1)=327.05

### EXAMPLES 24-38

The compounds in Table 3 below were prepared using a procedure similar to those employed in Example 23.

TABLE 3

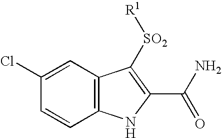

| Ex- | | | |
|---|---|---|---|
| am- | | | MS |
| ple | Name | R¹ | (M + 1) |
| 24 | 5-chloro-3-[(3,3,3- | CH₂CH₂CF₃ | 355.0 |
| | trifluoropropyl)sulfonyl]-1H- | | |
| | indole-2-carboxamide | | |
| 25 | 5-chloro-3-[(4,4,4- | CH₂CH₂CH₂CF₃ | 369.02 |
| | trifluorobutyl)sulfonyl]-1H- | | |
| | indole-2-carboxamide | | |
| 26 | 5-chloro-3-(cyclopropylsulfonyl)- 1H-indole-2-carboxamide | 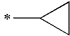 | 299.02 |
| 27 | 5-chloro-3- [(cyclopropylmethyl)sulfonyl]- 1H-indole-2-carboxamide | 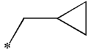 | 313.04 |
| 28 | 5-chloro-3-[(2- cyclopropylethyl)sulfonyl]-1H- indole-2-carboxamide | 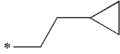 | 327.2 |
| 29 | 5-chloro-3-(cyclobutylsulfonyl)- 1H-indole-2-carboxamide | 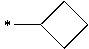 | 313.04 |
| 30 | 5-chloro-3-(cyclopentylsulfonyl)- 1H-indole-2-carboxamide | 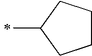 | 327.05 |
| 31 | 5-chloro-3-(cyclohexylsulfonyl)- 1H-indole-2-carboxamide | 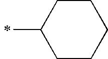 | 341.1 |
| 32 | 5-chloro-3-(cyclohex-2-en-1- ylsulfonyl)-1H-indole-2- carboxamide | 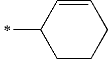 | 339.1 |
| 33 | 5-chloro-3- [(cyclohexylmethyl)sulfonyl]-1H- indole-2-carboxamide | 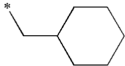 | 355.1 |
| 34 | 5-chloro-3-[(3,3,3-trifluoro-1- | CH(CH₃)CH₂CF₃ | 369.1 |
| | methylpropyl)sulfonyl]-1H- | | |
| | indole-2-carboxamide | | |
| 35 | methyl 1-{[2-(aminocarbonyl)-5- chloro-1H-indol-3- yl]sulfonyl} cyclobutanecarboxylate | 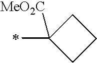 | 371.04 |
| 36 | ethyl 1-{[2-(aminocarbonyl)-5- chloro-1H-indol-3- yl]sulfonyl} cyclobutanecarboxylate | 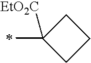 | 385.06 |
| 37 | 5-chloro-3-{[2,2,3,3- tetrafluorocyclobutyl)methyl] sulfonyl}-1H- indole-2-carboxamide | 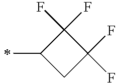 | 399.01 |
| 38 | 5-chloro-3-[(2,2,3,3- | CH₂CF₂CHF₂ | 373.0 |
| | tetrafluoropropyl)sulfonyl]-1H- | | |
| | indole-2-carboxamide | | |

### EXAMPLE 39

### 5-Chloro-3-[(3-methylbutyl)sulfonyl]-N-[(1-methyl-1H-imidazol-2-yl)methyl]-1-indole-2-carboxamid-indole-2-carboxamide

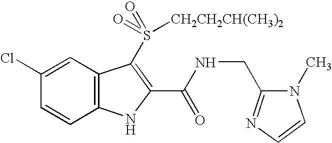

The title compound was prepared in the same way as described for 5-chloro-3-(cyclobutylmethylsulfonyl)-1-H-indole-2-carboxamide, except starting with ethyl 5-chloro-3-(3-methylbutylsulfonyl)-1-H-indole-2-carboxylate (50.0 mg, 0.140 mmol) and 2-aminomethyl-1-methylimidazole dihydrochloride salt (39 mg, 0.210 mmol) in methanol (1 mL) with triethylamine (0.2 mL). The title compound was isolated as the trifluoroacetate salt after purification by reverse phase HPLC. MS (M+1)=423

### EXAMPLE 40

### 5-Chloro-N-(1H-indazol-3-ylmethyl)-3-[(3-methylbutyl)sulfonyl]-1H-indole-2-carboxamide

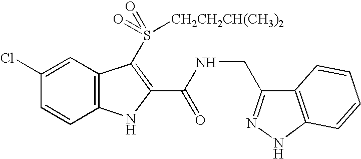

The title compound was prepared in the same way as described for 5-chloro-3-(cyclobutylmethylsulfonyl)-1-H-indole-2-carboxamide, except starting with ethyl 5-chloro-3-(3-methylbutylsulfonyl)-1-H-indole-2-carboxylate (50.0 mg, 0.140 mmol) and 3-aminomethylindazole (31 mg, 0.210 mmol) in methanol (1 mL). The title compound was isolated as the trifluoroacetate salt after purification by reverse phase HPLC. MS (M+1)=459

### EXAMPLE 41

### Encapsulated Oral Compositions

A capsule formulation suitable for use in the present invention can be prepared by filling standard two-piece gelatin capsules each with 100 mg of the compound of Example 1, 150 mg of lactose, 50 mg of cellulose, and 3 mg of stearic acid. Encapsulated oral compositions containing any one of the compounds of Examples 2 to 40 can be similarly prepared.

### EXAMPLE 42

### Assay for Inhibition of HIV Reverse Transcriptase

An assay to determine the in vivo inhibition of HIV reverse transcriptase by compounds of the present invention was conducted as follows: HIV-1 RT enzyme (1 DM) was combined with inhibitor or DMSO (10%) in assay buffer (50 mM Tris-HCl, pH 7.8, 1 mM dithiothreitol, 6 mM MgCl₂, 80 mM KCl, 0.025% CHAPS, 0.1 mM EGTA), and the mixture preincubated for 30 minutes at room temperature in microtiter Optiplates (Packard). 100 μL reaction mixtures were initiated with a combination of primer-template substrate (10 nM final concentration) and dNTPs (0.6 μM dNTPs, 0.75 μM [³H]-dGTP). The heterodimeric nucleic acid substrate was generated by annealing the DNA primer pD500 (described in Shaw-Reid et al., J. Biol. Chem., 278: 2777-2780; obtained from Integrated DNA Technologies) to t500, a 500 nucleotide RNA template created by in vitro transcription (see Shaw-Reid et al., J. Biol. Chem., 278: 2777-2780). After 1 hour incubation at 37° C., reactions were quenched by 10 μL streptavidin scintillation proximity assay beads (10 mg/mL, from Amersham Biosciences) in 0.5 M EDTA, pH 8. Microtiter plates were incubated an additional 10 minutes at 37° C. prior to quantification via Topcount (Packard). Representative compounds of the present invention exhibit inhibition of the reverse transcriptase enzyme in this assay. For example, the compounds set forth above in Examples 1 to 6 and 8 to 40 were tested in the assay and all were found to have IC₅₀ values of less than 10 micromolar. The compound of Example 7 was not tested.

Analogous assays were conducted substituting mutant HIV strains to determine the in vivo inhibition of compounds of the present invention against mutant HIV reverse transcriptase. In one strain the reverse transcriptase has the Y181C mutation and in the other strain the reverse transcriptase has the K103N mutation. The mutations were generated with the QUIKCHANGE site-directed mutagenesis kit (Stratagene). Certain compounds of the present invention exhibit inhibition of the reverse transcriptase enzyme in these assays. For example, the compounds set forth above in Examples 1, 8 10, 24 and 27-31 were found to have IC₅₀ values of less than 2 micromolar in the Y181C mutant assay, and the compounds of Examples 39 and 40 were found to have IC₅₀ values of less than 2 micromolar in the K103N mutant assay.

### EXAMPLE 43

### Assay for Inhibition of HIV Replication

An assay for the inhibition of acute HIV infection of T-lymphoid cells (alternatively referred to herein as the “spread assay”) was conducted in accordance with Vacca, J. P. et al., Proc. Natl. Acad. Sci. USA 1994, 91: 4096. Representative compounds of the present invention exhibit inhibition of HIV replication in this assay. For example, the compounds set forth in Examples 1, 3, 5, 8-13, 17, 18, 22-34, 37, 39 and 40 were found to have IC₉₅ values of less than or equal to 10 micromolar in the assay.

The compounds of Examples 2, 4, 6, 14-16, 19-21 and 38 were also tested in the spread assay up to 10 micromolar, but specific IC₉₅ values were not obtained; i.e., the IC₉₅ values for these compounds were greater than 10 micromolar. The compound of Example 7 was not tested.

### EXAMPLE 44

### Cytotoxicity

Cytotoxicity was determined by microscopic examination of the cells in each well in the spread assay, wherein a trained analyst observed each culture for any of the following morphological changes as compared to the control cultures: pH imbalance, cell abnormality, cytostatic, cytopathic, or crystallization (i.e., the compound is not soluble or forms crystals in the well). The toxicity value assigned to a given compound is the lowest concentration of the compound at which one of the above changes is observed. Representative compounds of the present invention that were tested in the spread assay (see Example 43) were examined for cytotoxicity. For those compounds for which an IC₉₅ value was determined in the spread assay, no cytotoxicity was exhibited at the IC₉₅ concentration; i.e., their toxicity value is greater than their IC₉₅ value. In particular, the compounds set forth in Examples 1, 3, 5, 8-13, 17, 18, 22-34, 37, 39 and 40 exhibited no cytotoxicity at their IC₉₅ concentrations.

While the foregoing specification teaches the principles of the present invention, with examples provided for the purpose of illustration, the practice of the invention encompasses all of the usual variations, adaptations and/or modifications that come within the scope of the following claims.

1. A compound of Formula I, or a pharmaceutically acceptable salt thereof: 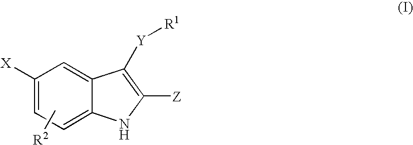
wherein:
X is:
(1) halogen,
(2) CN,
(3) NO₂,
(4) C(O)R^{A},
(5) C(O)OR^{A},
(6) C(O)N(R^{A})R^{B},
(7) SR^{A},
(8) S(O)R^{A},
(9) S(O)₂R^{A},
(10) S(O)₂N(R^{A})R^{B},
(11) N(R^{A})R^{B},
(12) N(R^{A})S(O)₂R^{B},
(13) N(R^{A})C(O)R^{B},
(14) N(R^{A})C(O)ORB,
(15) N(R^{A})S(O)₂N(R^{A})R^{B},
(16) OC(O)R^{A},
(17) OC(O)N(R^{A})R^{B},
(18) N(R^{A})C(O)N(R^{A})R^{B},
(19) C₁₋₆ alkyl,
(20) C₁₋₆ haloalkyl,
(21) C₂₋₆ alkenyl,
(22) C₂₋₆ alkynyl,
(23) OH,
(24) O—C₁₋₆ alkyl,
(26) O—C₁₋₆ haloalkyl, or
(27) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)R^{A}, OC(O)N(R^{A})R^{B}, or N(R^{A})C(O)N(R^{A})R^{B},
(28) C₃₋₈ cycloalkyl, or
(29) C₁₋₆ alkyl substituted with C₃₋₈ cycloalkyl;
Y is S, S(O), or S(O)₂;
Z is C(O)N(H)R^{C} or C(O)OR^{D};
R^{C} is:
(1) H,
(2) C₁₋₆ alkyl,
(3) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)R^{A}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule,
(4) O—C₁₋₆ alkyl,
(5) CycA,
(6) AryA,
(7) HetA,
(8) HetR, or
(9) C₁₋₆ alkyl substituted with CycA, AryA, HetA, or HetR;
R^{D} is:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)R^{A}, OC(O)N(R^{A})R^{B}, or N(R^{A})C(O)N(R^{A})R^{B}, with the proviso that the OH, O—C₁₋₆ alkyl, or O—C₁₋₆ haloalkyl is not attached to the carbon in C₁₋₆ alkyl that is directly attached to the rest of the molecule, or
(3) C₁₋₆ alkyl substituted with CycA, AryA, HetA, or HetR;
CycA is C₃₋₈ cycloalkyl which is optionally substituted with a total of from 1 to 6 substituents, wherein:
(i) from zero to 6 substituents are each independently:
(1) halogen,
(2) CN
(3) C₁₋₆ alkyl,
(4) OH,
(5) O—C₁₋₆ alkyl,
(6) C₁₋₆ haloalkyl, or
(7) O—C₁₋₆ haloalkyl, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
AryA is aryl which is optionally substituted with a total of from 1 to 6 substituents, wherein:
(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) halogen,
(8) CN,
(9) NO₂,
(10) N(R^{A})R^{B},
(11) C(O)N(R^{A})R^{B},
(12) C(O)R^{A},
(13) C(O)—C₁₋₆ haloalkyl,
(14) C(O)OR^{A},
(15) OC(O)R^{A},
(16) OC(O)N(R^{A})R^{B},
(17) SR^{A},
(18) S(O)R^{A},
(19) S(O)₂R^{A},
(20) S(O)₂N(R^{A})R^{B},
(21) N(R^{A})S(O)₂R^{B},
(22) N(R^{A})S(O)₂N(R^{A})R^{B},
(23) N(R^{A})C(O)R^{B},
(24) N(R^{A})C(O)N(R^{A})R^{B},
(25) N(R^{A})C(O)—C(O)N(R^{A})R^{B}, or
(26) N(R^{A})CO₂R^{B}, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
HetA is heteroaryl which is optionally substituted with a total of from 1 to 6 substituents, wherein:
(i) from zero to 6 substituents are each independently:
(1) C₁₋₆ alkyl,
(2) C₁₋₆ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, N(R^{A})C(O)N(R^{A})R^{B}, or N(R^{A})C(O)C(O)N(R^{A})R^{B},
(3) O—C₁₋₆ alkyl,
(4) C₁₋₆ haloalkyl,
(5) O—C₁₋₆ haloalkyl,
(6) OH,
(7) oxo,
(8) halogen,
(9) CN,
(10) NO₂,
(11) N(R^{A})R^{B},
(12) C(O)N(R^{A})R^{B},
(13) C(O)R^{A},
(14) C(O)—C₁₋₆ haloalkyl,
(15) C(O)OR^{A},
(16) OC(O)R^{A},
(17) OC(O)N(R^{A})R^{B},
(18) SR^{A},
(19) S(O)R^{A},
(20) S(O)₂R^{A},
(21) S(O)₂N(R^{A})R^{B},
(22) N(R^{A})S(O)₂R^{B},
(23) N(R^{A})S(O)₂N(R^{A})R^{B},
(24) N(R^{A})C(O)R^{B},
(25) N(R^{A})C(O)N(R^{A})R^{B},
(26) N(R^{A})C(O)—C(O)N(R^{A})R^{B}, or
(27) N(R^{A})CO₂R^{B}, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
HetR is a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, and wherein the saturated or mono-unsaturated heterocyclic ring is optionally substituted with a total of from 1 to 4 substituents, wherein:
(i) from zero to 4 substituents are each independently halogen, CN, C₁₋₆ alkyl, OH, oxo, C(O)R^{A}, C(O)₂R^{A}, S(O)R^{A}, SR^{A}, S(O)₂R^{A}, O—C₁₋₁₆ alkyl, C₁₋₆ haloalkyl, C₁₋₆ alkylene-CN, C₁₋₆ alkylene-OH, or C₁₋₆ alkylene-O—C₁₋₆ alkyl; and
(ii) from zero to 2 substituents are each independently CycE, HetE, AryE, HetF, or C₁₋₆ alkyl substituted with CycE, AryE, HetE, or HetF;
R¹ is:
(1) C₁₋₈ alkyl,
(2) C₁₋₈ alkyl substituted with OH, O—C₁₋₆ alkyl, O—C₁₋₆ haloalkyl, CN, NO₂, N(R^{A})R^{B}, C(O)N(R^{A})R^{B}, C(O)R^{A}, CO₂R^{A}, SR^{A}, S(O)R^{A}, S(O)₂R^{A}, S(O)₂N(R^{A})R^{B}, N(R^{A})C(O)R^{B}, N(R^{A})CO₂R^{B}, N(R^{A})S(O)₂R^{B}, N(R^{A})S(O)₂N(R^{A})R^{B}, OC(O)N(R^{A})R^{B}, or N(R^{A})C(O)N(R^{A})R^{B},
(3) C₁₋₈ haloalkyl,
(4) C₂₋₈ alkenyl,
(5) CycB,
(6) HetS, or
(7) C₁₋₈ alkyl substituted with CycB or HetT;
CycB is C₃₋₈ cycloalkyl or C₅₋₈ cycloalkenyl, wherein the cycloalkyl or cycloalkenyl is optionally substituted with a total of from 1 to 6 substituents, wherein:
(i) from zero to 6 substituents are each independently:
(1) halogen,
(2) CN
(3) C₁₋₆ alkyl,
(4) OH,
(5) O—C₁₋₆ alkyl,
(6) C₁₋₆ haloalkyl,
(7) O—C₁₋₆ haloalkyl, or
(8) C(O)OR^{A}, and
(ii) from zero to 2 substituents are each independently:
(1) CycE,
(2) AryE,
(3) O-AryE,
(4) HetE,
(5) HetF, or
(6) C₁₋₆ alkyl substituted with CycE, AryE, O-AryE, HetE, or HetF;
HetS is a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, wherein the saturated or mono-unsaturated heterocyclic ring is attached to the rest of the molecule via a ring carbon, and wherein the saturated or mono-unsaturated heterocyclic ring is optionally substituted with a total of from 1 to 4 substituents, wherein:
(i) from zero to 4 substituents are each independently halogen, CN, C₁₋₆ alkyl, OH, oxo, S(O)R^{A}, SR^{A}, S(O)₂R^{A}, O—C₁₋₆ alkyl, C(O)O—C₁₋₆ alkyl, C₁₋₆ haloalkyl, C₁₋₆ alkylene-CN, C₁₋₆ alkylene-OH, or C₁₋₆ alkylene-O—C₁₋₆ alkyl; and
(ii) from zero to 2 substituents are each independently CycE, HetE, AryE, HetF, or
C₁₋₆ alkyl substituted with CycE, AryE, HetE, or HetF;
HetT independently has the same definition as HetR;
R² is H or independently has the same definition as X;
each aryl is independently (i) phenyl, (ii) a 9- or 10-membered bicyclic, fused carbocylic ring system in which at least one ring is aromatic, or (iii) an 11- to 14-membered tricyclic, fused carbocyclic ring system in which at least one ring is aromatic;
each heteroaryl is independently (i) a 5- or 6-membered heteroaromatic ring containing from 1 to 4 heteroatoms independently selected from N, O and S, wherein each N is optionally in the form of an oxide, or (ii) a 9- or 10-membered bicyclic, fused ring system containing from 1 to 4 heteroatoms independently selected from N, O and S, wherein either one or both of the rings contain one or more of the heteroatoms, at least one ring is aromatic, each N is optionally in the form of an oxide, and each S in a ring which is not aromatic is optionally S(O) or S(O)₂;
each CycE is independently C₃₋₈ cycloalkyl which is optionally substituted with from 1 to 4 substituents each of which is independently halogen, C₁₋₆ alkyl, OH, O—C₁₋₆ alkyl, C₁₋₆ haloalkyl, or O—C₁₋₆ haloalkyl;
each AryE is independently phenyl or naphthyl, wherein the phenyl or naphthyl is optionally substituted with from 1 to 5 substituents each of which is independently halogen, CN, NO₂, C₁₋₆ alkyl, C₁₋₆ haloalkyl, OH, O—C₁₋₆ alkyl, O—C₁₋₆ haloalkyl, C(O)N(R^{A})R^{B}, C(O)R^{A}, CO₂R^{A}, SR^{A}, S(O)R^{A}, SO₂R^{A}, SO₂N(R^{A})R^{B}, or SO₂N(R^{A})C(O)R^{B};
each HetE is independently a 5- or 6-membered heteroaromatic ring containing from 1 to 4 heteroatoms independently selected from N, O and S, wherein each N is optionally in the form of an oxide, and wherein the heteroaromatic ring is optionally substituted with from 1 to 4 substituents each of which is independently halogen, C₁₋₆ alkyl, C₁₋₆ haloalkyl, O—C₁₋₆ alkyl, O—C₁₋₆ haloalkyl, OH, N(R^{A})R^{B}, N(R^{A})C(O)N(R^{A})R^{B}, or N(R^{A})CO₂R^{B};
each HetF is independently a 4- to 7-membered, saturated or mono-unsaturated heterocyclic ring containing at least one carbon atom and from 1 to 4 heteroatoms independently selected from N, O and S, where the S is optionally oxidized to S(O) or S(O)₂, and wherein the saturated or mono-unsaturated heterocyclic ring is optionally substituted with a total of from 1 to 4 substituents, each of which is independently halogen, CN, C₁₋₆ alkyl, OH, oxo, O—C₁₋₆ alkyl, C₁₋₆ haloalkyl, or O—C₁₋₆ haloalkyl;
each R^{A} is independently H or C₁₋₆ alkyl; and
each R^{B} is independently H or C₁₋₆ alkyl;
and with the proviso that:
(A) when:
(i) X is Cl, F, Br, NO₂, CN, OH, O—C₁₋₃ alkyl, NH₂, N(H)—C₁₋₃ alkyl, N(C₁₋₃ alkyl)₂, NHSO₂—C₁₋₃ alkyl, or NHC(O)—C₁₋₃ alkyl,
(ii) R² is H, and
(iii) Z is:
(a) C(O)N(H)R^{C}, wherein R^{C} is:
(1) H,
(2) C₁₋₆ alkyl,
(3) O—C₁₋₅ alkyl,
(4) C₁₋₅ alkyl substituted with OH, O—C₁₋₅ alkyl, OC(O)H, OC(O)—C₁₋₃ alkyl, CO₂H, C(O)O—C₁₋₃ alkyl, NH₂, N(H)—C₁₋₃ alkyl, N(C₁₋₃ alkyl)₂, C₃₋₆ cycloalkyl, AryA, HetA, or HetR, or
(5) C₃₋₆ cycloalkyl, AryA, HetA, or HetR, or
(b) C(O)OR^{D}, wherein R^{D} is:
(1) C₁₋₅ alkyl,
(2) O—C₁₋₅ alkyl, or
(3) C₁₋₅ alkyl substituted with OH, O—C₁₋₅ alkyl, AryA, HetA, or HetR,
then R¹ is not:
(1) C₁₋₈ alkyl,
(2) C₁₋₅ alkyl substituted with OH or O—C₁₋₅ alkyl, or
(3) HetS.

2. A compound according to claim 1, or a pharmaceutically acceptable salt thereof, wherein:
X is halogen;
Y is S, S(O), or S(O)₂;
Z is C(O)NH₂, C(O)NH—C₁₋₆ alkyl, or C(O)O—C₁₋₆ alkyl;
R¹ is:
(1) C₁₋₆ haloalkyl,
(2) CycB, or
(3) C₁₋₆ alkyl substituted with CycB; and
R² is H.

3. A compound according to claim 2, or a pharmaceutically acceptable salt thereof, which is a compound of Formula I-A: 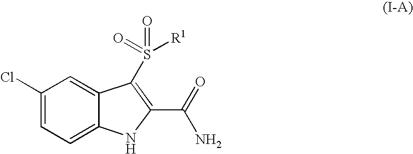
wherein:
R¹ is:
(1) C₁₋₆ fluoroalkyl,
(2) CycB, or
(3) CH₂-CycB, CH₂CH₂-CycB, or CH(CH₃)-CycB; and
CycB is C₃₋₆ cycloalkyl or C₅₋₆ cycloalkenyl, wherein the cycloalkyl or cycloalkenyl is optionally substituted with a total of from 1 to 4 substituents, each of which is independently Cl, Br, F, C₁₋₄ alkyl, O—C₁₋₄ alkyl, CF₃, or C(O)O—C₁₋₄ alkyl.

4. A compound according to claim 2, or a pharmaceutically acceptable salt thereof, which is a compound of Formula I-B: 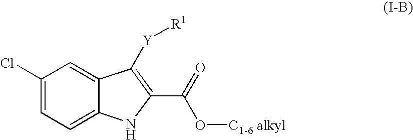
wherein:
Y is S or S(O)₂;
R¹ is:
(1) C₁₋₆ fluoroalkyl,
(2) CycB, or
(3) CH₂-CycB, CH₂CH₂-CycB, or CH(CH₃)-CycB; and
CycB is C₃₋₆ cycloalkyl or C₅₋₆ cycloalkenyl, wherein the cycloalkyl or cycloalkenyl is optionally substituted with a total of from 1 to 4 substituents, each of which is independently Cl, Br, F, C₁₋₄ alkyl, O—C₁₋₄ alkyl, CF₃, or C(O)O—C₁₋₄ alkyl.

5. A compound according to claim 1, or a pharmaceutically acceptable salt thereof, which is a compound selected from the group consisting of:
ethyl 5-chloro-3-(cyclobutylmethylthio)-1H-indole-2-carboxylate;
ethyl 5-chloro-3-[(2,2,2-trifluoroethyl)thio]-1H-indole-2-carboxylate;
ethyl 5-chloro-3-[(3,3,3-trifluoropropyl)thio]-1H-indole-2-carboxylate;
ethyl 5-chloro-3-[(4,4,4-trifluorobutyl)thio]-1H-indole-2-carboxylate;
ethyl 5-chloro-3-[(2,2,3,3,3-pentafluoropropyl)thio]-1H-indole-2-carboxylate;
ethyl 5-chloro-3-(cyclopropylthio)-1H-indole-2-carboxylate;
ethyl 5-chloro-3-[(cyclopropylmethyl)thio]-1H-indole-2-carboxylate;
ethyl 5-chloro-3-[(2-cyclopropylethyl)thio]-1H-indole-2-carboxylate;
ethyl 5-chloro-3-(cyclobutylthio)-1H-indole-2-carboxylate;
ethyl 5-chloro-3-(cyclopentylthio)-1H-indole-2-carboxylate;
ethyl 5-chloro-3-[(cyclohexylmethyl)thio]-1H-indole-2-carboxylate;
ethyl 5-chloro-3-(cyclohexylthio)-1H-indole-2-carboxylate;
ethyl 5-chloro-3-(cyclobutylmethylsulfonyl)-1H-indole-2-carboxylate;
ethyl 5-chloro-3-[(3,3,3-trifluoropropyl)sulfonyl]-1H-indole-2-carboxylate;
ethyl 5-chloro-3-[(4,4,4-trifluorobutyl)sulfonyl]-1H-indole-2-carboxylate;
ethyl 5-chloro-3-(cyclopropylsulfonyl)-1H-indole-2-carboxylate;
ethyl 5-chloro-3-[(cyclopropylmethyl)sulfonyl]-1H-indole-2-carboxylate;
ethyl 5-chloro-3-[(2-cyclopropylethyl)sulfonyl]-1H-indole-2-carboxylate;
ethyl 5-chloro-3-(cyclobutylsulfonyl)-1H-indole-2-carboxylate;
ethyl 5-chloro-3-(cyclopentylsulfonyl)-1H-indole-2-carboxylate;
ethyl 5-chloro-3-(cyclohexylsulfonyl)-1H-indole-2-carboxylate;
ethyl 5-chloro-3-[(cyclohexylmethyl)sulfonyl]-1H-indole-2-carboxylate;
5-chloro-3-(cyclobutylmethylsulfonyl)-1H-indole-2-carboxamide;
5-chloro-3-[(3,3,3-trifluoropropyl)sulfonyl]-1H-indole-2-carboxamide;
5-chloro-3-[(4,4,4-trifluorobutyl)sulfonyl]-1H-indole-2-carboxamide;
5-chloro-3-(cyclopropylsulfonyl)-1H-indole-2-carboxamide;
5-chloro-3-[(cyclopropylmethyl)sulfonyl]-1H-indole-2-carboxamide;
5-chloro-3-[(2-cyclopropylethyl)sulfonyl]-1H-indole-2-carboxamide;
5-chloro-3-(cyclobutylsulfonyl)-1H-indole-2-carboxamide;
5-chloro-3-(cyclopentylsulfonyl)-1H-indole-2-carboxamide;
5-chloro-3-(cyclohexylsulfonyl)-1H-indole-2-carboxamide;
5-chloro-3-(cyclohex-2-en-1-ylsulfonyl)-1H-indole-2-carboxamide;
5-chloro-3-[(cyclohexylmethyl)sulfonyl]-1H-indole-2-carboxamide;
5-chloro-3-[(3,3,3-trifluoro-1-methylpropyl)sulfonyl]-1H-indole-2-carboxamide;
methyl 1-{[2-(aminocarbonyl)-5-chloro-1H-indol-3-yl]sulfonyl}cyclobutanecarboxylate;
ethyl 1-{[2-(aminocarbonyl)-5-chloro-1H-indol-3-yl]sulfonyl}cyclobutanecarboxylate;
5-chloro-3-{[(2,2,3,3-tetrafluorocyclobutyl)methyl]sulfonyl}-1H-indole-2-carboxamide; and
5-chloro-3-[(2,2,3,3-tetrafluoropropyl)sulfonyl]-1H-indole-2-carboxamide.

6. A compound of Formula II, or a pharmaceutically acceptable salt thereof: 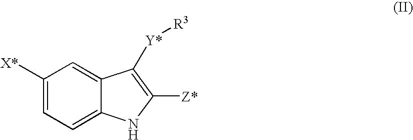
wherein:
X* is halogen;
Y* is S, S(O), or S(O)₂;
R³ is C₁₋₆ alkyl;
Z* is C(O)NHR⁴, wherein R⁴ is C₁₋₆ alkyl substituted with HetQ; and
HetQ is:
(i) a 5-membered heteroaromatic ring containing from 1 to 3 heteroatoms independently selected from 1 to 3 N atoms, from zero to 1 O atom, and from zero to 1 S atom, wherein the heteroaromatic ring is optionally substituted with from 1 to 2 substituents each of which is independently:
(1) C₁₋₆ alkyl,
(2) C₁₋₆ alkyl substituted with OH or O—C₁₋₆ alkyl,
(3) O—C₁₋₆ alkyl,
(4) C₁₋₆ haloalkyl,
(5) O—C₁₋₆ haloalkyl,
(6) OH,
(7) Cl, Br, or F,
(8) CN,
(9) C(O)N(H)—C₁₋₆ alkyl,
(10) C(O)N(C₁₋₆ alkyl)₂,
(11) S(O)₂—C₁₋₆ alkyl,
(12) S(O)₂NH₂,
(13) S(O)₂N(H)—C₁₋₆ alkyl,
(14) S(O)₂N(C₁₋₆ alkyl)₂,
(15) C₃₋₆ cycloalkyl which is optionally substituted with C₁₋₆ alkyl or phenyl,
(16) phenyl which is optionally substituted with from 1 to 3 substituents each of which is independently C₁₋₆ alkyl, O—C₁₋₆ alkyl, C₁₋₆ haloalkyl, O—C₁₋₆ haloalkyl, OH, halogen, NO₂, C(O)N(H)C₁₋₆ alkyl, C(O)N(C₁₋₆ alkyl)₂, CO₂—C₁₋₆ alkyl, or S(O)₂—C₁₋₆ alkyl,
(17) phenyl substituted with a saturated heterocyclic ring selected from the group consisting of: 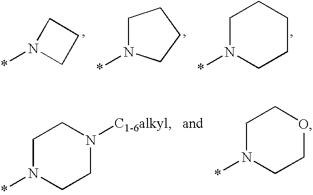
wherein the asterisk denotes the point of attachment to the rest of the molecule,
(18) C₁₋₆ alkyl substituted with C₃₋₆ cycloalkyl, or
(19) C₁₋₆ alkyl substituted with phenyl or O-phenyl, wherein the phenyl is optionally substituted with from 1 to 3 substituents each of which is independently C₁₋₆ alkyl, O—C₁₋₆ alkyl, C₁₋₆ haloalkyl, O—C₁₋₆ haloalkyl, OH, halogen, NO₂, C(O)N(H)C₁₋₆ alkyl, C(O)N(C₁₋₆ alkyl)₂, CO₂—C₁₋₆ alkyl, or S(O)₂—C₁₋₆ alkyl, or
(ii) a 5-membered heteroaromatic ring containing from 1 to 2 heteroatoms independently selected from 1 to 2 N atoms, from zero to 1 O atom, and from zero to 1 S atom, wherein the heteroaromatic ring has fused thereto a 6-membered carbocyclic ring that is saturated or partially or fully unsaturated, wherein the fused ring system is optionally substituted with from 1 to 4 substituents each of which is independently
(1) C₁₋₆ alkyl,
(2) C₁₋₆ alkyl substituted with OH or O—C₁₋₆ alkyl,
(3) O—C₁₋₆ alkyl,
(4) C₁₋₆ haloalkyl,
(5) O—C₁₋₆haloalkyl,
(6) OH,
(7) Cl, Br, or F,
(8) CN,
(9) C(O)N(H)—C₁₋₆ alkyl,
(10) C(O)N(C₁₋₆ alkyl)₂,
(11) S(O)₂—C₁₋₆ alkyl,
(12) S(O)₂NH₂,
(13) S(O)₂N(H)—C₁₋₆ alkyl, or
(14) S(O)₂N(C₁₋₆ alkyl)₂,
(15) C₃₋₆ cycloalkyl which is optionally substituted with C₁₋₆ alkyl or phenyl,
(16) phenyl which is optionally substituted with from 1 to 3 substituents each of which is independently C₁₋₆ alkyl, O—C₁₋₆ alkyl, C₁₋₆ haloalkyl, O—C₁₋₆ haloalkyl, OH, halogen, NO₂, C(O)N(H)C₁₋₆ alkyl, C(O)N(C₁₋₆ alkyl)₂, CO₂—C₁₋₆ alkyl, or S(O)₂—C₁₋₆ alkyl,
(17) phenyl substituted with a saturated heterocyclic ring selected from the group consisting of: 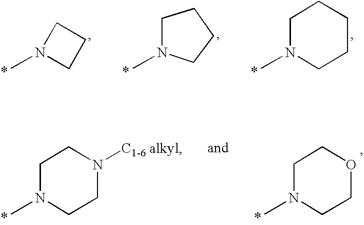
wherein the asterisk denotes the point of attachment to the rest of the molecule,
(18) C₁₋₆ alkyl substituted with C₃₋₆ cycloalkyl, or
(19) C₁₋₆ alkyl substituted with phenyl or O-phenyl, wherein the phenyl is optionally substituted with from 1 to 3 substituents each of which is independently C₁₋₆ alkyl, O—C₁₋₆ alkyl, C₁₋₆ haloalkyl, O—C₁₋₆ haloalkyl, OH, halogen, NO₂, C(O)N(H)C₁₋₆ alkyl, C(O)N(C₁₋₆ alkyl)₂, CO₂—C₁₋₆ alkyl, or S(O)₂—C₁₋₆ alkyl.

7. A compound according to claim 6, or a pharmaceutically acceptable salt thereof, wherein:
X* is chloro or bromo;
Y* is S or S(O)₂;
R³ is C₁₋₅ alkyl;
R⁴ is (CH₂)₁₋₃-HetQ or CH(CH₃)-HetQ;
HetQ is: 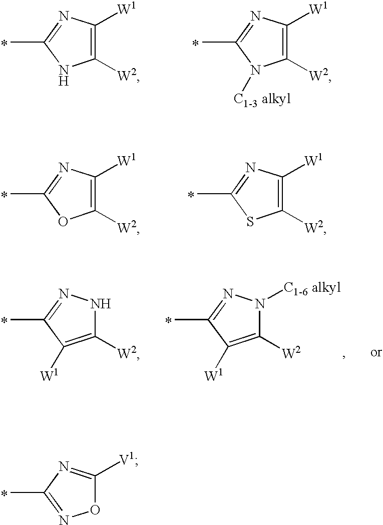
V¹ is:
(1) H,
(2) C₁₋₃ alkyl,
(3) C₁₋₃ alkyl substituted with OH or OCH₃,
(4) C₃₋₆ cycloalkyl which is optionally substituted with C₁₋₄ alkyl or phenyl,
(5) phenyl which is optionally substituted with from 1 to 3 substituents each of which is independently CH₃, OCH₃, CF₃, OCF₃, OH, Cl, Br, F, CN, NO₂, C(O)N(H)CH₃, C(O)N(CH₃)₂, CO₂CH₃, or S(O)₂CH₃,
(6) phenyl substituted with a saturated heterocyclic ring selected from the group consisting of: 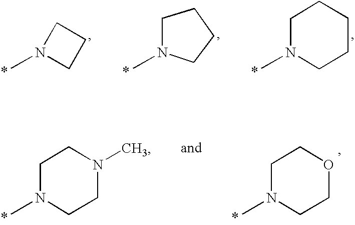
wherein the asterisk denotes the point of attachment to the rest of the molecule,
(7) CH₂-cyclopropyl,
(8) CH₂-phenyl, or
(9) CH₂—O-phenyl; and
W¹ and W² each independently have the same definition as V¹;
or alternatively, W¹ and W² together with the carbon atoms to which each is attached form a benzo ring.

8. A compound according to claim 7, or a pharmaceutically acceptable salt thereof, wherein:
X* is chloro;
Y* is S(O)₂;
R³ is C₁₋₅ alkyl;
R⁴ is CH₂-HetQ; and
HetQ is:

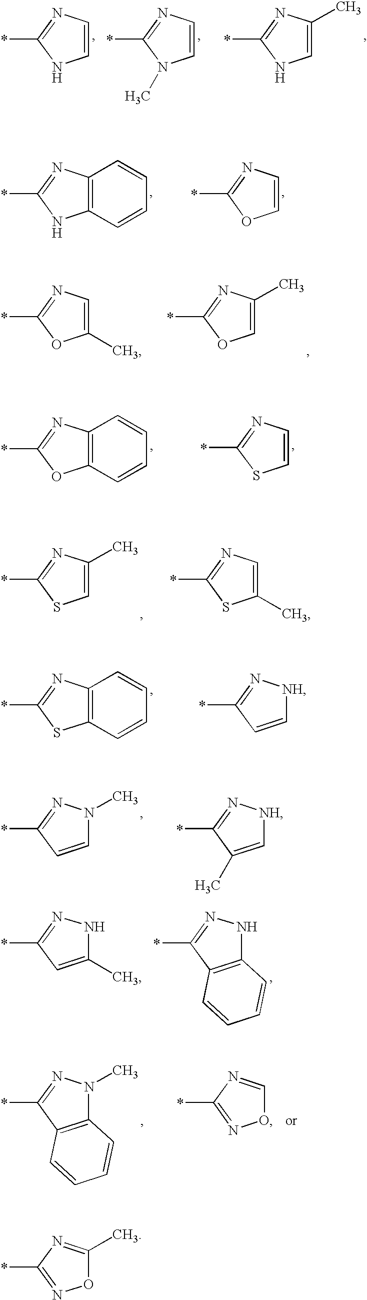

9. A compound according to claim 8, or a pharmaceutically acceptable salt thereof, which is a compound selected from the group consisting of:
5-chloro-3-[(3-methylbutyl)sulfonyl]-N-[(1-methyl-1H-imidazol-2-yl)methyl]-1-indole-2-carboxamide-indole-2-carboxamide; and
5-chloro-N-(1H-indazol-3-ylmethyl)-3-[(3-methylbutyl)sulfonyl]-1H-indole-2-carboxamide.

10. A pharmaceutical composition comprising an effective amount of a compound according to claim 1, or a pharmaceutically acceptable salt thereof, and a pharmaceutically acceptable carrier.

11. (canceled)

12. A method for the treatment of HIV infection, or the treatment or delay in the onset of AIDS, wherein the method comprises administering to a subject in need thereof an effective amount of a compound of Formula I, or a pharmaceutically acceptable salt thereof, as defined in claim 1.

13. (canceled)

14. (canceled)

15. A pharmaceutical composition comprising an effective amount of a compound according to claim 6, or a pharmaceutically acceptable salt thereof, and a pharmaceutically acceptable carrier.

16. A method for the treatment of HIV infection or for the treatment or delay in the onset of AIDS, wherein the method comprises administering to a subject in need thereof an effective amount of a compound according to claim 6, or a pharmaceutically acceptable salt thereof.
